(19) United States
(12) Patent Application Publication
(10) Pub. No.: US 2023/0398229 A1
(43) Pub. Date: Dec. 14, 2023

(54) ANTIBODY DRUG CONJUGATES COMPRISING STING AGONISTS, COMBINATIONS AND METHODS OF USE

(71) Applicant: Mersana Therapeutics, Inc., Cambridge, MA (US)

(72) Inventors: Raghida A. BUKHALID, Medford, MA (US); Naniye Cetinbas, Somerville, MA (US); Marc I. Damelin, Needham, MA (US); Timothy B. Lowinger, Carlisle, MA (US); Dorin Toader, Cambridge, MA (US)

(21) Appl. No.: 18/179,699

(22) Filed: Mar. 7, 2023

Related U.S. Application Data
(60) Provisional application No. 63/329,680, filed on Apr. 11, 2022.
(60) Provisional application No. 63/317,472, filed on Mar. 7, 2022.

(51) Int. Cl. A61K 47/68; A61K 39/395; A61P 35/00; C07K 16/32; C07K 16/28

(52) U.S. Cl. CPC A61K 47/6803; A61K 47/6855; A61K 39/39558; A61P 35/00; A61K 2039/545; C07K 16/2818; A61K 39/3955; C07K 2317/24; C07K 16/32

(57) ABSTRACT

The present disclosure provides combinations comprising HER2-targeted STING agonist antibody-drug conjugates and HER2-targeted therapies or immunotherapies. The present disclosure also provides uses of the combinations in treatment, e.g., treatment of cancer.

### RELATED APPLICATIONS

This application claims priority to, and the benefit of U.S. Provisional Application No. 63/317,472 filed Mar. 7, 2022, and U.S. Provisional Application No. 63/329,680 filed Apr. 11, 2022. The contents of each of these applications are hereby incorporated by reference in their entireties.

### REFERENCE TO AN ELECTRONIC SEQUENCE LISTING

The contents of the electronic sequence listing (MRSN_037_001US_SeqList_ST26.xml; Size: 18,657 bytes; and Date of Creation: Mar. 1, 2023) are herein incorporated by reference in its entirety.

### BACKGROUND

Stimulator of interferon genes (STING) is a receptor in the endoplasmic reticulum that propagates innate immune sensing of cytosolic pathogen derived- and self-DNA. STING is a 378 amino acid protein, which mainly contains three structural domains: (i) N-terminal transmembrane domain (aa 1-154); (ii) central globular domain (aa 155-341); and (iii) C-terminal tail (aa 342-379). STING may form symmetrical dimers combined with its ligands in V-shaped conformation, while not completely covering the bound ligands. A STING agonist can bind into the pocket region of STING. However, the STING activation process is easily inhibited in some severe disease conditions, resulting in the inactivation of the STING pathway. Therefore, screening and designing potent STING agonists is of great importance for cancer immune therapy and other infectious diseases treatments, including, but not limited to, obesity, liver injury, sugar-lipid metabolism, and virus infection. Specific targeting of immune pathways presents opportunities for cancer therapy, potentially offering greater specificity than cell population-based therapeutic approaches.

Antibody-drug conjugates (ADCs) are comprised of a drug like small molecule, covalently linked to an antibody. The antibody represents a targeting mechanism tuned to a specific site of action. Upon reaching the site, the ADC is designed to release a small molecule, the drug, allowing it to perform its designed function in a targeted manner, as opposed to diffusing systemically through the entire body of the subject. This targeted approach allows for treatment with drugs that would otherwise require doses so high as to be toxic when administered systemically and minimizes potential for on-target, off-tumor toxicity.

A key feature of the innate immune system is the recognition and elimination of foreign substances. Identification of these pathogenic invaders occurs through host recognition of evolutionarily conserved microbial structures known as pathogen-associated molecular patterns (PAMPs). Host recognition may occur by multiple pathways, such as activation of pattern recognition receptors (PRRs), which ultimately lead to downstream signaling events and culminate in the mounting of an immune response.

The antibody-drug conjugates of this disclosure modulate the activity of STING, and accordingly, may provide a beneficial therapeutic impact in treatment of diseases, disorders and/or conditions wherein modulation of STING (Stimulator of Interferon Genes) is beneficial, including, but not limited to, inflammation, allergic and autoimmune diseases, infectious diseases, cancer, pre-cancerous syndromes, and as vaccine adjuvants. In addition, combination therapy in which two or more drugs are used in certain dosing regimen or administration form, can enhance potency by exploiting additive or synergistic effects in the biological activity of the two or more drugs. There remains a need for new immunotherapies for the treatment of diseases, in particular cancer.

### SUMMARY

In some aspects, the present disclosure provides, inter alia, a combination therapy comprising at least one HER2-targeted STING agonist antibody-drug conjugate and at least one HER2-targeted therapy or at least one immunotherapy (e.g., an immune checkpoint inhibitor), wherein the conjugate comprises an antibody or antigen binding fragment thereof that specifically binds to an epitope of the human HER2 receptor.

In some aspects, the HER2-targeted STING agonist antibody-drug conjugate is a conjugate of Formula (A):

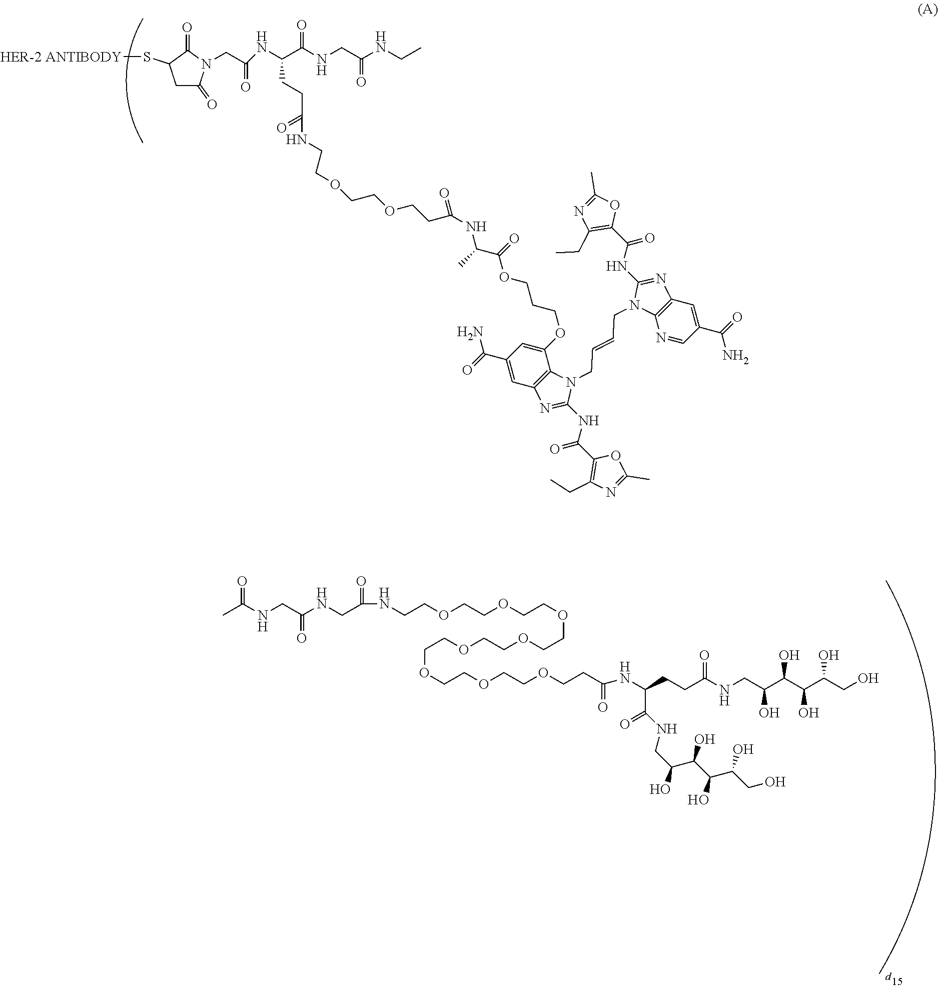

wherein the conjugate comprises a HER2 antibody comprising a variable heavy chain complementarity determining region 1 (CDRH1) comprising the amino acid sequence FTFSSYSMN (SEQ ID NO: 5); a variable heavy chain complementarity determining region 2 (CDRH2) comprising the amino acid sequence YISSSSSTIYYADSVKG (SEQ ID NO: 6); a variable heavy chain complementarity determining region 3 (CDRH3) comprising the amino acid sequence GGHGYFDL (SEQ ID NO: 7); and a variable light chain complementarity determining region 1 (CDRL1) comprising the amino acid sequence RASQSVSSSYLA (SEQ ID NO: 12); a variable light chain complementarity determining region 2 (CDRL2) comprising the amino acid sequence GASSRAT (SEQ ID NO: 13); and a variable light chain complementarity determining region 3 (CDRL3) comprising the amino acid sequence QQYHHSPLT (SEQ ID NO: 14), and d15 is about 8.

In some aspects, the HER2 antibody or antigen binding fragment thereof that specifically binds to an epitope of the human HER2 receptor includes residues 452 to 531 of the extracellular domain of the human HER2 receptor, residues 474 to 553 of SEQ ID NO: 1 or residues 452 to 531 of SEQ ID NO: 16.

In some aspects, the present disclosure provides compositions comprising the combination therapy comprising at least one HER2-targeted STING agonist antibody-drug conjugate and at least one HER2-targeted therapy or at least one at least one immunotherapy (e.g., an immune checkpoint inhibitor).

In some aspects, the HER2-targeted STING agonist antibody-drug conjugate enhances the efficacy of the HER2-targeted therapy or the immunotherapy (e.g., an immune checkpoint inhibitor).

In some aspects, the present disclosure provides, a composition comprising at least one HER2-targeted STING agonist antibody-drug conjugate and at least one HER2-targeted therapy.

In some aspects, the present disclosure provides, a composition comprising at least one HER2-targeted STING agonist antibody-drug conjugate and at least one immunotherapy (e.g., an immune checkpoint inhibitor).

In some aspects, the HER2-targeted therapy is an antibody or antigen binding fragment thereof that specifically binds HER2, a HER2-targeted antibody-drug conjugate that specifically binds HER2 or a small molecule inhibitor of HER2.

In some aspects, the present disclosure provides compositions comprising at least one HER2-targeted STING agonist antibody-drug conjugate and at least one antibody or antigen binding fragment thereof that specifically binds HER2, at least one HER2-targeted STING agonist antibody-drug conjugate that specifically binds HER2 or at least one small molecule inhibitor of HER2.

In some aspects, the HER2-targeted therapy is a HER2 antibody, a HER2 dimerization inhibitor antibody or a combination of a HER2 antibody and a HER2 dimerization inhibitor antibody.

In some aspects, the HER2-targeted therapy is trastuzumab, pertuzumab, a combination thereof or margetuximab or a biosimilar thereof.

In some aspects, the HER2-targeted therapy is trastuzumab, pertuzumab, or a combination thereof.

In some aspects, the HER2-targeted therapy is margetuximab or a biosimilar thereof.

In some aspects, the HER2-targeted therapy is a HER2-targeted antibody-drug conjugate that specifically binds HER2, such as, for example, ado-trastuzumab emtansine (T-DM1) (Kadcyla®) or fam-trastuzumab deruxtecan (trastuzumab deruxtecan) (Enhertu®).

In some aspects, the combination comprises at least one HER2-targeted STING agonist antibody-drug conjugate in combination with ado-trastuzumab emtansine (T-DM1) (Kadcyla®) or fam-trastuzumab deruxtecan (trastuzumab deruxtecan) (Enhertu®).

In some aspects, the HER2-targeted therapy is a small molecule inhibitor of HER2, such as, for example, tucatinib, neratinib or lapatinib.

In some aspects, the combination comprises at least one HER2-targeted STING agonist antibody-drug conjugate of the disclosure in combination with tucatinib, neratinib or lapatinib.

In some aspects, the combination comprising a HER2-targeted STING agonist antibody-drug conjugate of the disclosure is administered in combination with at least one antibody or antigen binding fragment thereof that specifically binds HER2, at least one HER2-targeted STING agonist antibody-drug conjugate that specifically binds HER2 or at least one small molecule inhibitor of HER2.

In some aspects, the immune checkpoint inhibitor suitable for combination and the methods of this disclosure is a monoclonal antibody, a humanized antibody, a fully human antibody, a fusion protein or a combination thereof.

In some aspects, the immune checkpoint inhibitor suitable for the combination and the methods of the disclosure is a PD-1 inhibitor or a PD-L1 inhibitor.

In some aspects, the combination comprises a HER2-targeted STING agonist antibody-drug conjugate of the disclosure in combination with a PD-1 inhibitor or a PD-L1 inhibitor.

In some aspects, the combination comprises a HER2-targeted STING agonist antibody-drug conjugate of the disclosure in combination with an immune checkpoint inhibitor, such as, for example, avelumab, durvalumab, dostarlimab, pembrolizumab, cemiplimab, nivolumab, or atezolizumab. In some aspects, the combination comprises a HER2-targeted STING agonist antibody-drug conjugate of the disclosure in combination with dostarlimab. In some aspects, the combination comprises a HER2-targeted STING agonist antibody-drug conjugate of the disclosure in combination with pembrolizumab.

In some aspects, the combination comprising a HER2-targeted STING agonist antibody-drug conjugate of the disclosure is administered in combination with an immune checkpoint inhibitor, such as, for example, avelumab, durvalumab, dostarlimab, pembrolizumab, cemiplimab, nivolumab, or atezolizumab. In some aspects, the combination comprising a HER2-targeted STING agonist antibody-drug conjugate of the disclosure is administered in combination with dostarlimab. In some aspects, the combination comprising a HER2-targeted STING agonist antibody-drug conjugate of the disclosure is administered in combination with pembrolizumab.

The combination of HER2-targeted STING agonist antibody-drug conjugates and HER2-targeted therapies or immune checkpoint inhibitors are useful in treating pathologies such as, for example, a cancer in a subject. For example, the combinations comprising HER2-targeted STING agonist antibody-drug conjugates and at least one HER2-targeted therapy or at least one immune checkpoint inhibitor disclosed herein are useful in treating, preventing, delaying the progression of or otherwise ameliorating a symptom of a cancer in a subject.

In some embodiments, the cancer is, for example, selected from the group consisting of anal cancer, astrocytoma, leukemia, lymphoma, head and neck cancer, liver cancer, testicular cancer, cervical cancer, sarcoma, hemangioma, esophageal cancer, eye cancer, laryngeal cancer, mouth cancer, mesothelioma, skin cancer, myeloma, oral cancer, rectal cancer, colorectal cancer, throat cancer, bladder cancer, breast cancer, urothelial cancer, uterine cancer, ovarian cancer, prostate cancer, lung cancer, non-small cell lung cancer (NSCLC), colon cancer, pancreatic cancer, renal cancer, gastric cancer and gastric esophagogastric junction cancer.

In some aspects, the combination therapy disclosed herein is useful in treating, preventing, delaying the progression of or otherwise ameliorating a symptom of breast cancer, a symptom of gastric cancer, a symptom of gastric esophagogastric junction cancer, a symptom of non-small cell lung cancer (NSCLC) or a symptom of colorectal cancer in a subject.

In some aspects, the combination therapy disclosed herein is useful in treating, preventing, delaying the progression of or otherwise ameliorating a symptom of breast cancer, gastric cancer, gastric esophagogastric junction cancer, non-small cell lung cancer (NSCLC) or colorectal cancer in a subject.

In some aspects, the combination therapy disclosed herein is useful in treating, preventing, delaying the progression of or otherwise ameliorating a symptom of breast cancer, in a subject. In some aspects, the combination therapy disclosed herein is useful in treating, preventing, delaying the progression of or otherwise ameliorating a symptom of HER2+ breast cancer, in a subject. In some aspects, the combination therapy disclosed herein is useful in treating, preventing, delaying the progression of or otherwise ameliorating a symptom of HER2− breast cancer, in a subject. In some aspects, the combination therapy disclosed herein is useful in treating, preventing, delaying the progression of or otherwise ameliorating a symptom of metastatic HER2+ breast cancer, in a subject. In some aspects, the combination therapy disclosed herein is useful in treating, preventing, delaying the progression of or otherwise ameliorating a symptom of metastatic HER2+ breast cancer, in a subject who has received at least one, at least two, at least three, or at least four prior lines of breast cancer therapy. In some aspects, the combination therapy disclosed herein is useful in treating, preventing, delaying the progression of or otherwise ameliorating a symptom of metastatic HER2+ breast cancer, in a subject who has received three or more prior lines of breast cancer therapy.

In some aspects, the combination comprising a HER2-targeted STING agonist antibody-drug conjugate and at least one HER2-targeted therapy or at least one immune checkpoint inhibitor used in any of the aspect of the methods and uses provided herein can be administered at any stage of the disease. For example, such a combination therapy can be administered to a patient suffering cancer of any stage, from early to metastatic.

A combination therapy comprising a HER2-targeted STING agonist antibody-drug conjugate and at least one HER2-targeted therapy or at least one immune checkpoint inhibitor used in any of the aspect of these methods and uses can be administered either without another therapeutic agent, or in combination with one or more chemotherapeutic agents or other agents. In some aspects, the additional agent is any of the toxins described herein. In some aspects, the additional agent is (1) an EGFR inhibitor (e.g., tyrosine kinase inhibitors or targeted anti-EGFR antibodies), (2) a BRAF inhibitor, (3) an ALK inhibitor, (4) a hormone receptor inhibitor, (5) a mTOR inhibitor, (6) a VEGF inhibitor, or (7) a cancer vaccine. In some aspects, the additional agent is a standard, first line chemotherapeutic agent, such as, for example, ado-trastuzumab emtansine (Kadcyla), lapatinib, anastrozole, letrozole, exemestane, everolimus, fulvestrant, tamoxifen, toremifene, megestrol acetate, fluoxymesterone, ethinyl estradiol, paclitaxel, capecitabine, gemcitabine, eribulin, vinorelbine, cyclophosphamide, carboplatin, docetaxel, albumin-bound paclitaxel, cisplatin, epirubicin, ixabepilone, doxorubicin, fluorouracil, oxaliplatin, fluoropyrimidine, irinotecan, ramucirumab, mitomycin, leucovorin, cetuximab, bevacizumab, erlotinib, afatinib, crizotinib, permetrexed, ceritinib, etoposide, vinblastine, vincristine, ifosfamid, liposomal doxorubicin, topotecan, altretamine, melphalan or leuprolide acetate. In some aspects, the additional agent is Kadcyla (ado-trastuzumab emtansine).

In some aspects, the combination comprising HER2-targeted STING agonist antibody-drug conjugates and HER2-targeted therapies or immune checkpoint inhibitors and additional agent(s) is formulated into a single therapeutic composition, and the components are administered simultaneously. Alternatively, the HER2-targeted STING agonist antibody-drug conjugate, HER2-targeted therapy or immune checkpoint inhibitor and additional agent, if any, are separate from each other, e.g., each is formulated into a separate therapeutic composition, and can be administered simultaneously, or at different times during a treatment regimen. For example, the HER2-targeted STING agonist antibody-drug is administered prior to the administration of the HER2-targeted therapy or immune checkpoint inhibitor combination; the HER2-targeted STING agonist antibody-drug is administered after the administration of the HER2-targeted therapy or immune checkpoint inhibitor combination. As described herein, the HER2-targeted STING agonist antibody-drug and the HER2-targeted therapy or the immune checkpoint inhibitor combination is administered in single doses or in multiple doses.

Pharmaceutical compositions according to the disclosure can include a suitable carrier. These pharmaceutical compositions can be included in kits, such as, for example, diagnostic kits.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### BRIEF DESCRIPTION OF THE FIGURES

FIG. 1 is a graph showing the anti-tumor efficacy of Conjugate 1, trastuzumab, a combination of Conjugate 2 and trastuzumab, and a combination of Conjugate 1 and trastuzumab, in SKOV3 tumor-bearing mice at varying dose levels and dosing regimens.

FIG. 2 is a graph showing the anti-tumor efficacy of Conjugate 1; a combination of Conjugate 1 and trastuzumab; a combination of Conjugate 1 and pertuzumab; a combination of Conjugate 1, trastuzumab, and pertuzumab; a combination of Conjugate 2 and trastuzumab; a combination of Conjugate 2 and pertuzumab; and a combination of Conjugate 2, trastuzumab, and pertuzumab in JIMT-1 tumor-bearing mice at varying dose levels and dosing regimens.

FIG. 3 is a graph showing the anti-tumor efficacy of Conjugate 1; Conjugate 2; trastuzumab; pertuzumab; a combination of trastuzumab and pertuzumab; a combination of Conjugate 1 and trastuzumab; a combination of Conjugate 1 and pertuzumab; and a combination of Conjugate 1, trastuzumab, and pertuzumab in SNU-5 tumor-bearing mice at varying dose levels and dosing regimens.

FIG. 4 is a graph showing the anti-tumor efficacy of Conjugate 1; Conjugate 2; Enhertu; and a combination of Conjugate 1 and Enhertu in JIMT-1 tumor-bearing mice at varying dose levels and dosing regimens.

FIG. 5 is a graph showing the anti-tumor efficacy of Conjugate 3; Conjugate 4; anti-PD-1 RMP1-14; a combination of Conjugate 3 and anti-PD-1 RMP1-14; and a combination of Conjugate 4 and anti-PD-1 RMP1-14 in EMT6-RHER2 MSA tumor-bearing mice at varying dose levels and dosing regimens.

FIGS. 6A and 6B are graphs showing the tumor volumes of mice previously treated with Conjugate 4 when rechallenged with EMT-6-MSA cells or CT26 colon/colorectal cancer cells respectively. FIGS. 6C and 6D are graphs showing the tumor volumes of mice previously treated with Conjugate 4 and anti-PD-1 RMP1-14 when rechallenged with EMT-6-MSA cells or CT26 colon/colorectal cancer cells respectively.

FIG. 7 is a series of graphs depicting PD-L1 expression in murine and human SKOV3 tumors treated with vehicle, a control ADC, and Conjugate 1 (HER2-targeted STING agonist ADC).

### DETAILED DESCRIPTION

The present disclosure provides novel HER2-targeted STING agonist antibody-drug conjugates, pharmaceutical compositions containing them, and various uses of the conjugates.

### Definitions

The chemical names provided for the intermediate compounds and/or the compounds of this disclosure described herein may refer to any one of the tautomeric representations of such compounds (in some instances, such alternate names are provided with the experimental). It is to be understood that any reference to a named compound (an intermediate compound or a compound of the disclosure) or a structurally depicted compound (an intermediate compound or a compound of the disclosure) is intended to encompass all tautomeric forms including zwitterionic forms of such compounds and any mixture thereof.

It is to be understood that the terms “In some aspect”, “In some aspect of the present disclosure”, and “In some aspect of a compound of the present disclosure” may be used interchangeably where appropriate.

The term “about”, “approximately”, or “approximate”, when used in connection with a numerical value, means that a collection or range of values is included. In some aspects, “about X” includes a range of values that are ±25%, ±20%, ±15%, ±10%, ±5%, ±2%, ±1%, ±0.5%, ±0.2%, or ±0.1% of X, where X is a numerical value. In some aspects, the term “about” refers to a range of values which are 5% more or less than the specified value. In some aspects, the term “about” refers to a range of values which are 2% more or less than the specified value. In some aspects, the term “about” refers to a range of values which are 1% more or less than the specified value.

Recitation of ranges of values are merely intended to serve as a shorthand method of referring individually to each separate value falling within the range, unless otherwise indicated herein, and each separate value is incorporated into the specification as if it were individually recited herein. A range used herein, unless otherwise specified, includes the two limits of the range. In some aspects, the expressions “x being an integer between 1 and 6” and “x being an integer of 1 to 6” both mean “x being 1, 2, 3, 4, 5, or 6”, i.e., the terms “between X and Y” and “range from X to Y, are inclusive of X and Y and the integers there between.

As used herein, the terms “HER2” (also known as ErbB-2, NEU, HER-2, and CD340), when used herein, refers to human epidermal growth factor receptor 2 (SwissProt P04626) and includes any variants, isoforms and species homologs of HER2 which are naturally expressed by cells, including tumor cells, or are expressed on cells transfected with the HER2 gene. Species homologs include rhesus monkey HER2 (macaca mulatta; Genbank accession No. GI:109114897). These terms are synonymous and may be used interchangeably.

As used herein, the term “HER2 antibody” or “anti-HER2 antibody” is an antibody which binds specifically to the antigen HER2.

The term “antibody” as used herein, is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. The numbering of the antibody amino acids is according to Kabat EU Index (See Kabat, E. A., et al., Sequences of Protein of immunological interest, Fifth Edition, US Department of Health and Human Services, US Government Printing Office (1991)).

The term “antibody fragment” refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

The term “antibody that binds to the same epitope” as a reference antibody as used herein, refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

When used herein in the context of two or more antibodies, the term “competes with” or “cross-competes with” indicates that the two or more antibodies compete for binding to HER2, An antibody “blocks” or “cross-blocks” one or more other antibodies from binding to HER2 if the antibody competes with the one or more other antibodies 25% or more, with 25%-74% representing “partial block” and 75%-400% representing “full block”. Unless otherwise defined or negated by context, the terms “competes with”, “cross-competes with”, “blocks” or “cross-blocks” when used herein is also intended to cover such pairs of antibodies.

The term “epitope” refers to the particular site on an antigen molecule to which an antibody binds.

The term “independently”, as used herein, means that where more than one substituent is selected from a number of possible substituents, those substituents may be the same or different.

The term “pharmaceutically acceptable”, as used herein, refers to those compounds, conjugates, materials, compositions, and/or dosage forms which are, within the scope of sound medical judgment, suitable for use in contact with the tissues of human beings and animals without excessive toxicity, irritation, or other problem or complication, commensurate with a reasonable benefit/risk ratio.

The term “pharmaceutical composition” as used herein, refers to a mixture, formulation, or solution comprising at least one therapeutic agent to be administered to a subject, e.g., a mammal or human, in order or treat a particular disease or condition affecting the subject. The present pharmaceutical combinations can be formulated in suitable pharmaceutical compositions for enteral or parenteral administration, such as sugar-coated tablets, tablets, capsules or suppositories, or ampoules. If not indicated otherwise, these are prepared in a manner known per se, for example by means of various conventional mixing, comminution, direct compression, granulating, sugar-coating, dissolving, lyophilizing processes, or fabrication techniques readily apparent to those skilled in the art. It will be appreciated that the unit content of a combination partner contained in an individual dose of each dosage form need not in itself constitute an effective amount since the necessary effective amount may be reached by administration of a plurality of dosage units. One of ordinary skill in the art may select one or more of the aforementioned carriers with respect to the particular desired properties of the dosage form by routine experimentation and without any undue burden. The amount of each carriers used may vary within ranges conventional in the art. The pharmaceutical compositions provided herein may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model.

As used herein, the term “preventing,” “prevent,” or “protecting against” describes reducing or eliminating the onset of the symptoms or complications of such disease, condition or disorder.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at the start of treatment or may become resistant during treatment. In some embodiments, the subject in need thereof received and failed all known effective therapies for a disease or disorder disclosed herein. In some embodiments, the subject in need thereof received at least one prior therapy.

The term “therapeutically effective amount” refers to an amount of an active compound or pharmaceutical agent, including a conjugate of the disclosure, which elicits the biological or medicinal response in a tissue system, animal or human that is being sought by a researcher, veterinarian, medical doctor or other clinician, which includes alleviation or partial alleviation of the symptoms of the disease, syndrome, condition, or disorder being treated.

An “effective amount” is intended to mean that amount of a conjugate that, when administered to a patient in need of such treatment, is sufficient to effectively treat or prevent, as defined herein. The amount of a given conjugate that will correspond to such an amount will vary depending upon factors such as the particular conjugate (e.g., the potency (pICso), efficacy (EC₅₀), and the biological half-life of the particular conjugate), disease condition and its severity, the identity (e.g., age, size and weight) of the patient in need of treatment, but can nevertheless be routinely determined by one skilled in the art. Likewise, the duration of treatment and the time period of administration (time period between dosages and the timing of the dosages, e.g., before/with/after meals) of the conjugate will vary according to the identity of the mammal in need of treatment (e.g., weight), the particular conjugate and its properties (e.g., pharmacokinetic properties), disease or disorder and its severity and the specific composition and method being used, but can nevertheless be determined by one of skill in the art.

The term “composition” refers to a product that includes the specified ingredients in therapeutically effective amounts, as well as any product that results, directly, or indirectly, from combinations of the specified ingredients in the specified amounts.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

The term “STING agonist”, as used herein, refers to a compound or moiety which is capable of interacting with STING, e.g., by binding to STING and/or inducing downstream signal transduction (e.g., characterized by activation of the molecules associated with STING function). This includes direct phosphorylation of STING, IRF3 and/or NF-kB and could also include STATE. In some aspects, STING pathway activation results in increased production of type 1 interferons (mainly IFN-α and IFN-b) and/or expression of interferon-stimulated genes.

The term “STING agonist drug moiety”, as used herein, refers to a moiety derived from a STING agonist and capable of interacting with STING. In some aspects, the STING agonist drug moiety is a moiety derived from a STING agonist to allow the moiety being linked to the rest of a conjugate of the present disclosure.

“Immunotherapy” as used herein, refers to an agent that that activates or suppresses the immune system or a component of the immune system. Exemplary immunotherapies include, but are not limited to, monoclonal antibodies, immune checkpoint inhibitors, vaccines, cytokine therapy, adoptive cellular therapies, or immune system modulators.

“Immune checkpoint inhibitor” or “immune checkpoint inhibiting agent” or “immune checkpoint blocking agent” or “immune checkpoint modulator” as used herein, refers to an agent that binds an inhibitory immune checkpoint protein and blocks its activity thereby enabling the immune system to recognize tumor cells and allowing a sustained immunotherapy response. The inhibition can be competitive or non-competitive inhibition that can be steric or allosteric. In cases where an immune checkpoint protein is an immune stimulating protein, an immune checkpoint inhibitor acts to promote the activity of the immune stimulating protein, such as by binding and activating the stimulatory immune checkpoint protein or by inhibiting by interfering with, such as by binding or deactivating, inhibitors of the stimulatory immune checkpoint protein. An example of an immune checkpoint inhibitor is an anti-immune checkpoint protein antibody.

“Immune checkpoints” as used herein refer to inhibitory pathways of the immune system that are responsible for maintaining self-tolerance and modulating the duration and amplitude of physiological immune responses in peripheral tissues in order to minimize collateral tissue damage. Immune checkpoints are regulated by immune checkpoint proteins.

“Immune checkpoint protein” as used herein, refers to a protein, for example, a receptor (e.g., CTLA4 or PD-1) or a ligand (e.g., PD-L1) that regulates or modulates the extent of an immune response. The immune checkpoint proteins can be inhibitory or stimulatory. In particular, the immune checkpoint proteins are inhibitory to the activation of the immune response. Thus, inhibition of an inhibitory immune checkpoint protein acts to stimulate or activate an immune response, such as T cell activation and proliferation.

“Combination Therapy” as used herein refers to a treatment in which a subject is given two or more therapeutic agents, such as at least two or at least three therapeutic agents, for treating a disease or disorder. For purposes herein, a combination therapy includes therapy with a HER2-targeted STING agonist antibody-drug conjugate and a HER2-targeted therapy or an immune checkpoint inhibitor.

As used herein “co-administration”, “co-administering” or “co-administered” refers to the administration of at least two different therapeutic agents sufficiently close in time. Such administration may be done in any order, including simultaneous administration, as well as temporally spaced order from a few seconds up to several days apart. Such administration may also include more than a single administration of one agent and/or independently the other agent. The administration of the agents may be by the same or different routes.

The term “simultaneous administration” as used herein in relation to the administration of medicaments refers to the administration of medicaments such that the individual medicaments are present within a subject at the same time. In addition to the concomitant administration of medicaments (via the same or alternative routes), simultaneous administration may include the administration of the medicaments (via the same or an alternative route) at different times.

As used herein, the terms “at least one” item or “one or more” item each include a single item selected from the list as well as mixtures of two or more items selected from the list.

As used herein, the term “immune response” relates to any one or more of the following: specific immune response, non-specific immune response, both specific and non-specific response, innate response, primary immune response, adaptive immunity, secondary immune response, memory immune response, immune cell activation, immune cell-proliferation, immune cell differentiation, and cytokine expression.

The conjugates of the disclosure are useful in methods for treating or ameliorating a viral infection, disease, a syndrome, a condition or a disorder that is affected by the agonism of STING. Such methods comprise, consist of and/or consist essentially of administering to a subject, including an animal, a mammal, and a human in need of such treatment, amelioration and/or prevention, a therapeutically effective amount of a conjugate of the disclosure, or an enantiomer, diastereomer, solvate or pharmaceutically acceptable salt thereof.

The terms “conjugate(s) of the disclosure” or “conjugate(s) of the present disclosure”, as used herein, mean a conjugate as defined herein, in any form, i.e., any tautomeric form, any isomeric form, any salt or non-salt form (e.g., as a free acid or base form, or as a salt, particularly a pharmaceutically acceptable salt thereof) and any physical form thereof (e.g., including non-solid forms (e.g., liquid or semi-solid forms), and solid forms (e.g., amorphous or crystalline forms, specific polymorphic forms, solvate forms, including hydrate forms (e.g., mono-, di- and hemi-hydrates)), and mixtures of various forms.

Accordingly, included within the present disclosure are the conjugates as disclosed herein, in any salt or non-salt form and any physical form thereof, and mixtures of various forms. While such are included within the present disclosure, it will be understood that the conjugates of the present disclosure, in any salt or non-salt form, and in any physical form thereof, may have varying levels of activity, different bioavailabilities and different handling properties for formulation purposes.

It is understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

### HER2 Antibodies

In some aspects, the HER2 antibodies suitable for conjugation bind the human HER2 in soluble form, or membrane bound (i.e., when expressed on a cell surface). In some aspects, the present disclosure provides monoclonal antibodies that bind HER2 and are humanized or fully human. In some aspects, the present disclosure provides monoclonal antibodies that bind HER2 specifically. These antibodies are collectively referred to herein as “HER2” antibodies.

In some aspects, the HER2 antibodies suitable for conjugation bind to a HER2 epitope with an equilibrium dissociation constant (K_{d} or K_{D}) of <1 μM (e.g., <100 nM; <10 nM; <1 nM). In some aspects, the present disclosure provides monoclonal antibodies that bind HER2 and are humanized or fully human. for example, the HER2 antibodies provided herein exhibit a K_{d} in the range approximately between <1 nM to about 1 pM.

In some aspects, the HER2 antibodies disclosed herein serve to modulate, block, inhibit, reduce, antagonize, neutralize, or otherwise interfere with the functional activity of HER2. In some aspects, functional activities of HER2 include for example, modulation of PI3K-Akt pathway activity. In some aspects, the HER2 antibodies completely or partially inhibit HER2 functional activity by partially or completely modulating, blocking, inhibiting, reducing antagonizing, neutralizing, or otherwise interfering with PI3K-Akt pathway activity. PI3K-Akt pathway activity is assessed using any art-recognized method for detecting PI3K-Akt pathway activity, including, but not limited to detecting levels of phosphorylated Akt in the presence and absence of an antibody or antigen binding fragment disclosed herein.

In some aspects, the HER2 antibodies are considered to completely modulate, block, inhibit, reduce, antagonize, neutralize, or otherwise interfere with HER2 functional activity when the level of HER2 functional activity in the presence of the HER2 antibody is decreased by at least 80%, e.g., by 81%, 82%, 83%, 84%, 85%, 86%, 87%, 88%, 89%, 90%, 91%, 92%, 93%, 94%, 95%, 96%, 97%, 98%, 99%, or 100% as compared to the level of HER2 functional activity in the absence of binding with a HER2 antibody described herein. In some aspects, the HER2 antibodies are considered to partially modulate, block, inhibit, reduce, antagonize, neutralize, or otherwise interfere with HER2 functional activity when the level of HER2 activity in the presence of the HER2 antibody is decreased by less than 95%, e.g., 10%, 20%, 25%, 30%, 40%, 50%, 60%, 75%, 80%, 85%, or 90% as compared to the level of HER2 activity in the absence of binding with a HER2 antibody described herein.

In some aspects, exemplary HER2 antibodies disclosed herein include, the XMT-1519 antibody. This antibody shows specificity for human HER2 and has been shown to inhibit the functional activity of HER2 in vitro.

In some embodiments, the antibodies or antigen-binding fragments thereof disclosed herein comprising the HER2 monoclonal antibody XMT-1519 includes a heavy chain (HC), heavy chain variable region (VH), light chain (LC), and a light chain variable region (VL), as shown in the amino acid and corresponding nucleic acid sequences presented in Table I below. The variable heavy chain region and variable light chain region for each antibody are shaded in the amino acid sequences below. The complementarity determining regions (CDRs) of the heavy chain and the light chain are underlined in the amino acid sequences presented below.

TABLE I
HER2 human or humanized monoclonal
antibody XMT-1519 sequences

| SEQ | |
|---|---|
| ID NO: | Sequence Description |
| 1 | Full-length human HER2 |
| | receptor |
| 2 | XMT-1519 Heavy chain variable |
| | region |
| 3 | XMT-1519 IgG1 Heavy chain |
| | constant region |
| 4 | XMT-1519 Heavy Chain Amino |
| | Acid Sequence |
| 5 | XMT-1519 CDRH1 |
| 6 | XMT-1519 CDRH2 |
| 7 | XMT-1519 CDRH3 |
| 8 | XMT-1519 Heavy Chain variable |
| | region nucleic acid sequence |
| 9 | XMT-1519 Light chain variable region |
| 10 | XMT-1519 Light chain constant region |
| 11 | XMT-1519 Light Chain Amino Acid Sequence |
| 12 | XMT-1519 CDRL1 |
| 13 | XMT-1519 CDRL2 |
| 14 | XMT-1519 CDRL3 |
| 15 | XMT-1519 Light Chain variable region nucleic |
| | acid sequence |
| 16 | Extracellular domain (ECD) of the human |
| | HER2 receptor |

Antibodies and antigen binding fragments thereof disclosed herein specifically bind to an epitope on the full-length human HER2 receptor comprising the amino acid sequence of SEQ ID NO: 1.

Antibodies and antigen binding fragments thereof disclosed herein specifically bind to an epitope on the extracellular domain (ECD) of the human HER2 receptor comprising the amino acid sequence of SEQ ID NO: 16.

In some embodiments, the antibodies of the present disclosure exhibit HER2 binding characteristics that differ from antibodies described in the art. In some embodiments, the antibodies disclosed herein bind to a different epitope of HER2, in that they cross-block each other but not trastuzumab, pertuzumab, Fab37, or chA21 from binding to HER2. Further, as opposed to the known antibodies, the antibodies disclosed herein can internalize efficiently into HER2-expressing cells without promoting cell proliferation.

In some embodiments, the antibodies disclosed herein are fully human monoclonal antibodies that bind to novel epitopes and/or have other favorable properties for therapeutic use. In some embodiments, exemplary properties include, but are not limited to, favorable binding characteristics to cancer cells expressing human HER2 at high or low levels, specific binding to recombinant human and cynomolgus monkey HER2, efficient internalization upon binding to HER2, high capacity for killing cancer cells expressing high or low levels of HER2 when administered as an antibody drug conjugate (ADC), no substantial agonistic effect on the proliferation of HER2-expressing cancer cells, and/or provide for effective antibody-dependent cellular cytotoxicity (ADCC)-mediated killing of HER2-expressing cells, as well as any combination of the foregoing properties.

In some embodiments, the antibodies disclosed herein also include an antibody or antigen binding fragment thereof that specifically binds to an epitope of the human HER2 receptor that includes residues 452 to 531 of the extracellular domain of the human HER2 receptor, residues 474 to 553 of SEQ ID NO: 1 or residues 452 to 531 of SEQ ID NO: 16.

In some embodiments, the antibodies disclosed herein include an antibody or an antigen binding fragment thereof that binds at least a portion of the N-terminus of domain IV of human HER2 receptor but does not cross-compete with an antibody that binds to epitope 4D5 of the human HER2 receptor. In some embodiments, the antibodies or antigen binding fragments thereof described herein do not cross-compete with trastuzumab for binding to the human HER2 receptor, as trastuzumab is known to bind epitope 4D5 of the human HER2 receptor. As used herein, the term epitope 4D5 of the human HER2 receptor refers to amino acid residues 529 to 627 of the extracellular domain of the human HER2 receptor, residues 551 to 649 of SEQ ID NO: 1 or residues 529 to 627 of SEQ ID NO: 16. In some embodiments, the antibody or antigen binding fragment thereof also binds at least one epitope on cynomolgus monkey HER2 receptor.

In some embodiments, the antibodies disclosed herein also include an antibody or antigen binding fragment thereof that specifically binds to an epitope of the human HER2 receptor that includes residues 452 to 500 of the extracellular domain of the human HER2 receptor, residues 474 to 522 of SEQ ID NO: 1 or residues 452 to 500 of SEQ ID NO: 16.

In some embodiments, the antibodies disclosed herein also include an antibody or antigen binding fragment thereof that specifically binds to an epitope of the human HER2 receptor that includes at least one of amino acid residue selected from amino acid residues E521, L525 and R530 of the extracellular domain of the human HER2 receptor, e.g., residues 543, 547, and 552 of SEQ ID NO: 1, and residues 521, 525, and 530 of SEQ ID NO: 16. In some embodiments, the antibodies disclosed herein include an antibody or antigen binding fragment thereof that specifically binds to an epitope of the extracellular domain of the human HER2 receptor that includes at least two amino acid residues selected from amino acid residues E521, L525 and R530 of the extracellular domain of the human HER2 receptor. In some embodiments, the antibodies disclosed herein also include an antibody or antigen binding fragment thereof that specifically binds to an epitope of the human HER2 receptor that includes at least amino acid residues E521, L525 and R530 of the extracellular domain of the human HER2 receptor. In some embodiments, any or all of these antibodies or antigen binding fragments thereof also bind at least one epitope on cynomolgus monkey HER2 receptor.

In some embodiments, antibodies disclosed herein also include an antibody or an antigen binding fragment thereof that binds to at least a portion of domain III and at least a portion of the N-terminus of domain IV of human HER2 receptor but does not cross-compete with Fab37 monoclonal antibody or an antibody that binds to epitope 4D5 of the human HER2 receptor. In some embodiments, the antibodies or antigen binding fragments thereof described herein do not cross-compete with the Fab37 monoclonal antibody and/or trastuzumab for binding to the human HER2 receptor. In some embodiments, the antibody or antigen binding fragment thereof also binds at least one epitope on cynomolgus monkey HER2 receptor.

In some embodiments, the antibodies disclosed herein also include an antibody or antigen binding fragment thereof that specifically binds to an epitope of the human HER2 receptor that includes residues 520 to 531 of the extracellular domain of the human HER2 receptor, residues 542 to 553 of SEQ ID NO: 1 or residues 520 to 531 of SEQ ID NO: 16.

In some embodiments, the antibodies disclosed herein also include an antibody or antigen binding fragment thereof that specifically binds to an epitope of the human HER2 receptor that includes at least one amino acid residue selected from residues C453, H456, H473, N476, R495, G496, H497, and W499 of the extracellular domain of the human HER2 receptor, e.g., residues 475, 478, 495, 498, 517, 518, 519, and 521 of SEQ ID NO: 1 or residues 453, 456, 473, 476, 495, 496, 497 and 499 of SEQ ID NO: 16. In some embodiments, the antibodies disclosed herein include an antibody or antigen binding fragment thereof that specifically binds to an epitope of the extracellular domain of the human HER2 receptor that includes at least two amino acid residues, at least three amino acid residues, at least four amino acid residues, at least five amino acid residues, or at least six amino acid residues selected from amino acid residues C453, H456, H473, N476, R495, G496, H497, and W499 of the extracellular domain of the human HER2 receptor. In some embodiments, the antibodies disclosed herein include an antibody or antigen binding fragment thereof that specifically binds to an epitope of the extracellular domain of the human HER2 receptor that includes at least amino acid residues C453, H456, H473, N476, R495, G496, H497, and W499 of the extracellular domain of the human HER2 receptor. In some embodiments, any or all of these antibodies or antigen binding fragments thereof also bind at least one epitope on cynomolgus monkey HER2 receptor.

In some embodiments, the antibodies disclosed herein also include an antibody or antigen binding fragment thereof that specifically binds to an epitope of the human HER2 receptor that includes at least one amino acid residue selected from residues C453, H473, N476, R495, H497, and W499 of the extracellular domain of the human HER2 receptor, e.g., residues 475, 495, 498, 517, 519, and 521 of SEQ ID NO: 1 or residues 453, 473, 476, 495, 497 and 499 of SEQ ID NO: 16. In some embodiments, the antibodies disclosed herein include an antibody or antigen binding fragment thereof that specifically binds to an epitope of the extracellular domain of the human HER2 receptor that includes at least two amino acid residues, at least three amino acid residues, at least four amino acid residues, at least five amino acid residues, or at least six amino acid residues selected from amino acid residues C453, H473, N476, R495, H497, and W499 of the extracellular domain of the human HER2 receptor. In some embodiments, the antibodies disclosed herein include an antibody or antigen binding fragment thereof that specifically binds to an epitope of the extracellular domain of the human HER2 receptor that includes at least amino acid residues C453, H473, N476, R495, H497, and W499 of the extracellular domain of the human HER2 receptor. In some embodiments, any or all of these antibodies or antigen binding fragments thereof also bind at least one epitope on cynomolgus monkey HER2 receptor.

In some embodiments, these antibodies show specificity for human HER2, and they have been shown to modulate, e.g., block, inhibit, reduce, antagonize, neutralize, or otherwise interfere with the PI3K-Akt pathway which promotes cell survival by reducing levels of phosphorylated AKT. In some embodiments, these antibodies internalize from the cell surface of HER2-expressing cells at a rate that is the same or substantially similar to the rate at which trastuzumab or a biosimilar thereof internalizes. In some embodiments, these antibodies and antigen binding fragments have a rate of internalization that is about 50% of the total surface bound at time 0 being internalized by 4 hours.

In some embodiments the antibodies disclosed herein comprise a heavy chain variable region having an amino acid sequence at least 80%, 81%, 82%, 83%, 84%, 85%, 86%, 87% 88%, 89%, 90%, 91%, 92%, 93%, 94%, 95%, 96%, 97% 98%, 99% or more identical to a sequence selected from SEQ ID NO: 2 and a light chain variable region having an amino acid sequence at least 80%, 81%, 82%, 83%, 84%, 85%, 86%, 87% 88%, 89%, 90%, 91%, 92%, 93%, 94%, 95%, 96%, 97% 98%, 99% or more identical to a sequence selected from SEQ ID NOs: 9.

In some embodiments, the antibodies disclosed herein comprise a heavy chain amino acid sequence at least 80%, 81%, 82%, 83%, 84%, 85%, 86%, 87% 88%, 89%, 90%, 91%, 92%, 93%, 94%, 95%, 96%, 97% 98%, 99% or more identical to the amino acid sequence of SEQ ID NO: 4 and a light chain amino acid sequence at least 80%, 81%, 82%, 83%, 84%, 85%, 86%, 87% 88%, 89%, 90%, 91%, 92%, 93%, 94%, 95%, 96%, 97% 98%, 99% or more identical to the amino acid sequence of SEQ ID NO: 11.

In some embodiments, the antibodies disclosed herein comprise the heavy chain variable region amino acid sequence of SEQ ID NO: 2 and the light chain variable region amino acid sequence of SEQ ID NO: 9.

In some embodiments, the antibodies disclosed herein comprise the heavy chain amino acid sequence of SEQ ID NO: 4 and the light chain amino acid sequence of SEQ ID NO: 11.

In some embodiments, the antibodies disclosed herein comprise the CDRH1 amino acid sequence of SEQ ID NO: 5, the CDRH2 amino acid sequence of SEQ ID NO: 6, the CDRH3 amino acid sequence of SEQ ID NO: 7, the CDRL1 amino acid sequence of SEQ ID NO: 12, the CDRL2 amino acid sequence of SEQ ID NO: 13, and the CDRL3 amino acid sequence of SEQ ID NO: 14.

In some embodiments, the antibodies disclosed herein include one or more conservative amino acid substitutions in a variable domain sequence such as 1, 2, 3, 4, 5, 6, 7, 8, 9, 10, 11, 12, 13, 14, 15, or more conservative substitutions in a variable domain sequence. In some embodiments, these conservative amino acid substitutions are in a CDR region, e.g., 1, 2, 3, 4, 5, 6, 7, 8, 9, 10, 11, 12, 13, 14, 15, or more conservative substitutions are made cumulatively across all CDRs. In some embodiments, up to 1, 2, 3, or 4 conservative amino acid substitutions may be present in each CDR sequence, e.g., SEQ ID NOs: 5-7 and 12-14.

Those skilled in the art will recognize that it is possible to determine, without undue experimentation, if a monoclonal antibody has the same specificity as a monoclonal antibody XMT-1519, by ascertaining whether the former prevents the latter from binding to a natural binding partner or other molecule known to be associated with HER2. In some embodiments, if the monoclonal antibody being tested competes with the monoclonal antibody disclosed herein, as shown by a decrease in binding by the monoclonal antibody disclosed herein, then the two monoclonal antibodies bind to the same, or a closely related, epitope.

In some embodiments, an alternative method for determining whether a monoclonal antibody has the specificity of monoclonal antibody disclosed herein is to pre-incubate the monoclonal antibody disclosed herein with soluble HER2 (with which it is normally reactive), and then add the monoclonal antibody being tested to determine if the monoclonal antibody being tested is inhibited in its ability to bind HER2. If the monoclonal antibody being tested is inhibited then, in all likelihood, it has the same, or functionally equivalent, epitopic specificity as the monoclonal antibody disclosed herein.

In some embodiments, screening of monoclonal antibodies disclosed herein, can be also carried out, e.g., by measuring HER2-mediated PI3K-Akt pathway activity, and determining whether the test monoclonal antibody is able to modulate, block, inhibit, reduce, antagonize, neutralize or otherwise interfere with PI3K-Akt pathway activity. In some embodiments, the HER2 antibodies suitable for conjugation can be generated and purified by well-known techniques e.g., WO 2015/195917 and PCT/US2018/019873, each of which is incorporated herein in its entirety by reference.

### HER2-Targeted STING Agonist Antibody Conjugates

The invention pertains to combination therapies involving immunoconjugates comprising an HER2-targeted antibody conjugated to a STING agonist.

In some embodiments, the present disclosure provides, inter alia, combination therapies comprising at least one HER2-targeted STING agonist antibody-drug conjugate and at least one HER2-targeted therapy or at least one immunotherapy (e.g., an immune checkpoint inhibitor), wherein the conjugate comprises an antibody or antigen binding fragment thereof that specifically binds to an epitope of the human HER2 receptor.

In some embodiments, the HER2-targeted STING agonist antibody-drug conjugate is a conjugate of Formula (A):

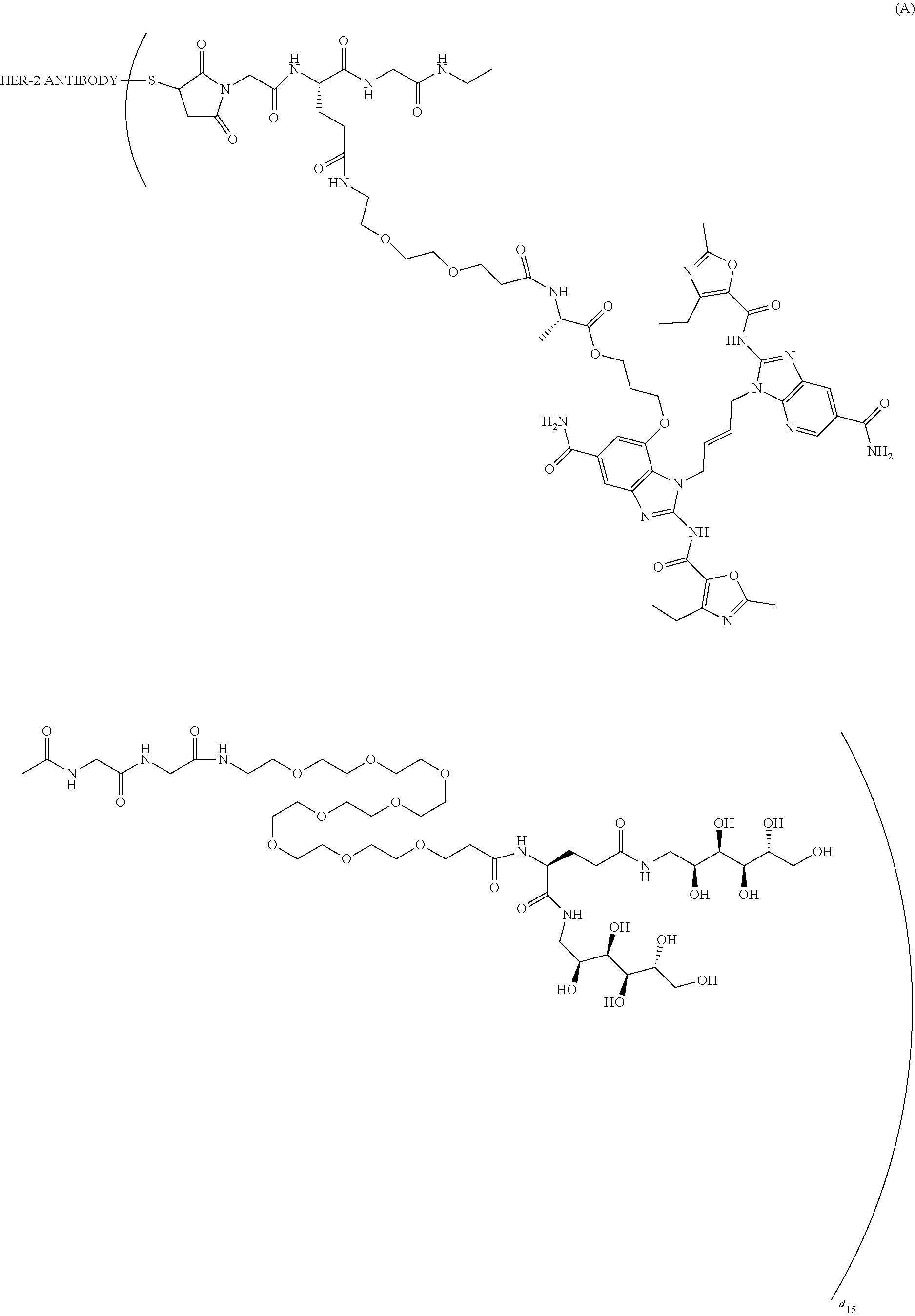

wherein the conjugate comprises a HER2 antibody comprising a variable heavy chain complementarity determining region 1 (CDRH1) comprising the amino acid sequence FTFSSYSMN (SEQ ID NO: 5); a variable heavy chain complementarity determining region 2 (CDRH2) comprising the amino acid sequence YISSSSSTIYYADSVKG (SEQ ID NO: 6); a variable heavy chain complementarity determining region 3 (CDRH3) comprising the amino acid sequence GGHGYFDL (SEQ ID NO: 7); and a variable light chain complementarity determining region 1 (CDRL1) comprising the amino acid sequence RASQSVSSSYLA (SEQ ID NO: 12); a variable light chain complementarity determining region 2 (CDRL2) comprising the amino acid sequence GASSRAT (SEQ ID NO: 13); and a variable light chain complementarity determining region 3 (CDRL3) comprising the amino acid sequence QQYHHSPLT (SEQ ID NO: 14), and d15 is about 8.

In some embodiments, the HER2 antibody or antigen binding fragment thereof that specifically binds to an epitope of the human HER2 receptor that includes residues 452 to 531 of the extracellular domain of the human HER2 receptor, residues 474 to 553 of SEQ ID NO: 1 or residues 452 to 531 of SEQ ID NO: 16.

In some embodiments, d15 is 2, 4, 6, or 8. In some embodiments, d15 is 6 or 8.

In some embodiments, d15 is 8. In some embodiments, d15 is 6.

### HER2-Targeted Therapy

Any suitable HER2-targeted therapy has been contemplated herein for use in the combinations and methods of the disclosure. In some embodiments, the HER2-targeted therapy is an antibody or antigen binding fragment thereof that specifically binds HER2, a second HER2-targeted STING agonist antibody-drug conjugate that specifically binds HER2 or a small molecule inhibitor of HER2.

In some embodiments, the HER2-targeted therapy is a HER2 antibody, a HER2 dimerization inhibitor antibody or a combination of a HER2 antibody and a HER2 dimerization inhibitor antibody.

In some embodiments, the HER2-targeted therapy is a HER2 antibody, a HER2 dimerization inhibitor antibody or a combination of a HER2 antibody and a HER2 dimerization inhibitor antibody. In some embodiments, the HER2 antibody, the HER2 dimerization inhibitor antibody or the combination of a HER2 antibody and the HER2 dimerization inhibitor antibody is trastuzumab, pertuzumab or margetuximab or biosimilars thereof. In some embodiments, the HER2-targeted therapy is trastuzumab or pertuzumab or a combination thereof. In some embodiments, the HER2-targeted therapy is trastuzumab. In some embodiments, the HER2-targeted therapy is pertuzumab. In some embodiments, the HER2-targeted therapy is margetuximab or a biosimilar thereof. In some embodiments, the HER2-targeted therapy is a biosimilar of trastuzumab or a biosimilar of pertuzumab or a combination of a biosimilar of trastuzumab and a biosimilar of pertuzumab. In some embodiments, the HER2-targeted therapy is a biosimilar of trastuzumab. In some embodiments, the HER2-targeted therapy is a biosimilar of pertuzumab. In some embodiments, the HER2-targeted therapy is a combination of a biosimilar of trastuzumab and a biosimilar of pertuzumab.

In some embodiments, the combination comprises at least one HER2-targeted STING agonist antibody-drug conjugate of the disclosure in combination with at least one HER2 antibody, at least one HER2 dimerization inhibitor antibody or a combination of at least one HER2 antibody and at least one HER2 dimerization inhibitor antibody. In some embodiments, the combination comprises at least one HER2-targeted STING agonist antibody-drug conjugate in combination with trastuzumab or pertuzumab or a combination thereof or margetuximab. In some embodiments, the combination comprises at least one HER2-targeted STING agonist antibody-drug conjugate in combination with trastuzumab or pertuzumab or a combination thereof. In some embodiments, the combination comprises at least one HER2-targeted STING agonist antibody-drug conjugate in combination with margetuximab. In some embodiments, the combination comprises at least one HER2-targeted STING agonist antibody-drug conjugate in combination with at least one biosimilar of trastuzumab or at least one biosimilar of pertuzumab or a combination thereof or at least one biosimilar of margetuximab. In some embodiments, the combination comprises at least one HER2-targeted STING agonist antibody-drug conjugate in combination with at least one biosimilar of trastuzumab or at least one biosimilar of pertuzumab or a combination thereof. In some embodiments, the combination comprises at least one HER2-targeted STING agonist antibody-drug conjugate in combination with at least one biosimilar of margetuximab.

In some embodiments, the combination comprising at least one HER2-targeted STING agonist antibody-drug conjugate of the disclosure is administered in combination with at least one HER2 antibody, at least one HER2 dimerization inhibitor antibody or a combination thereof or margetuximab. In some embodiments, the combination comprising at least one HER2-targeted STING agonist antibody-drug conjugate of the disclosure is administered in combination with at least one trastuzumab or at least one pertuzumab or a combination thereof or at least one margetuximab. In some embodiments, the combination comprising at least one HER2-targeted STING agonist antibody-drug conjugate of the disclosure is administered in combination with at least one biosimilar of trastuzumab or at least one biosimilar of pertuzumab or a combination thereof or at least one biosimilar of margetuximab.

In some embodiments, the HER2-targeted therapy is a HER2-targeted antibody-drug conjugate that specifically binds HER2. In some embodiments, the HER2-targeted antibody-drug conjugate is, for example, ado-trastuzumab emtansine (T-DM1) (Kadcyla®) fam-trastuzumab deruxtecan (trastuzumab deruxtecan) (Enhertu®) or a biosimilar thereof.

In some embodiments, the combination comprises at least one HER2-targeted STING agonist antibody-drug conjugate of the disclosure in combination with at least one HER2-targeted antibody-drug conjugate. In some embodiments, the combination comprises at least one HER2-targeted STING agonist antibody-drug conjugate of the disclosure in combination with ado-trastuzumab emtansine (T-DM1) (Kadcyla®) or fam-trastuzumab deruxtecan (trastuzumab deruxtecan) (Enhertu®). In some embodiments, the combination comprises at least one HER2-targeted STING agonist antibody-drug conjugate of the disclosure in combination with a biosimilar of ado-trastuzumab emtansine (T-DM1) (Kadcyla®) or a biosimilar of fam-trastuzumab deruxtecan (trastuzumab deruxtecan) (Enhertu®). In some embodiments, the combination comprises at least one HER2-targeted STING agonist antibody-drug conjugate of the disclosure in combination with ado-trastuzumab emtansine (T-DM1) (Kadcyla®) or a biosimilar thereof. In some embodiments, the combination comprises at least one HER2-targeted STING agonist antibody-drug conjugate of the disclosure in combination with fam-trastuzumab deruxtecan (trastuzumab deruxtecan). (Enhertu®) or a biosimilar of thereof.

In some embodiments, the combination comprising at least one HER2-targeted STING agonist antibody-drug conjugate of the disclosure is administered in combination with at least one HER2-targeted antibody-drug conjugate. In some embodiments, the combination comprising at least one HER2-targeted STING agonist antibody-drug conjugate of the disclosure is administered in combination with ado-trastuzumab emtansine (T-DM1) (Kadcyla®) or fam-trastuzumab deruxtecan (trastuzumab deruxtecan) (Enhertu®).

In some embodiments, the HER2-targeted therapy is a small molecule inhibitor of HER2. In some embodiments, the small molecule inhibitor of HER2 is, for example, tucatinib, neratinib or lapatinib. In some embodiments, the combination comprises at least one HER2-targeted STING agonist antibody-drug conjugate of the disclosure in combination with, at least one small molecule inhibitor of HER2. In some embodiments, the combination comprises at least one HER2-targeted STING agonist antibody-drug conjugate of the disclosure in combination with tucatinib, neratinib or lapatinib.

In some embodiments, the combination comprising at least one HER2-targeted STING agonist antibody-drug conjugate of the disclosure is administered in combination with, at least one small molecule inhibitor of HER2, such as, for example, tucatinib, neratinib or lapatinib.

In some embodiments, the HER2-targeted STING agonist antibody-drug conjugate enhances the efficacy of the HER2-targeted therapy.

### Immunotherapies

Any suitable immunotherapy has been contemplated herein for use in the combinations and methods of the disclosure. In some embodiments, the immunotherapy is an immune checkpoint inhibitor including, but not limited to, immune checkpoint molecule binding proteins, small molecule inhibitors, antibodies, antibody-derivatives (including Fab fragments and scFvs), antibody-drug conjugates, antisense oligonucleotides, siRNA, aptamers, peptides and peptide mimetics. Inhibitory nucleic acids that decrease the expression and/or activity of immune checkpoint molecules can also be used in the combinations and methods disclosed herein.

In some embodiments, the immune checkpoint inhibitor reduces the expression or activity of one or more immune checkpoint proteins. In another embodiments, the immune checkpoint inhibitor reduces the interaction between one or more immune checkpoint proteins and their ligands. See, e.g., US20160101128.

In some embodiments, the immune checkpoint inhibitor suitable for the combinations and methods of the disclosure is a monoclonal antibody, a humanized antibody, a fully human antibody, a fusion protein or a combination thereof.

In some embodiments, the composition comprises at least one HER2-targeted STING agonist antibody-drug conjugate of the disclosure in combination with at least one immune checkpoint inhibitor. In some embodiments, the composition comprises at least one HER2-targeted STING agonist antibody-drug conjugate of the disclosure in combination with a monoclonal antibody, a humanized antibody, a fully human antibody, a fusion protein or a combination thereof.

In some embodiments, the immune checkpoint inhibitor is a PD-1 inhibitor or a PD-L1 inhibitor.

In some embodiments, the PD-1 inhibitor or the PD-L1 inhibitor is pembrolizumab (MK-3475), nivolumab (BMS-936558), pidilizumab (CT-011), AMP-224, MDX-1 105, durvalumab (MEDI4736), MPDL3280A, BMS-936559, IPH2101, TSR-042, TSR-022, cemiplimab, ipilimumab, lirilumab, atezolizumab, avelumab, dostarlimab, tremelimumab, or a combination thereof.

In some embodiments, the immune checkpoint inhibitor is avelumab, durvalumab, dostarlimab, pembrolizumab, cemiplimab, nivolumab, or atezolizumab.

In some embodiments, the immune checkpoint inhibitor is pembrolizumab, dostarlimab, nivolumab, or atezolizumab. In some embodiments, the immune checkpoint inhibitor is pembrolizumab. In some embodiments, the immune checkpoint inhibitor is dostarlimab.

In some embodiments, the combination comprises at least one HER2-targeted STING agonist antibody-drug conjugate of the disclosure and at least one immune checkpoint inhibitor. In some embodiments, the combination comprises at least one HER2-targeted STING agonist antibody-drug conjugate of the disclosure and at least one PD-1 inhibitor or at least one PD-L1 inhibitor.

In some embodiments, the combination comprises at least one HER2-targeted STING agonist antibody-drug conjugate of the disclosure and avelumab, durvalumab, dostarlimab, pembrolizumab, cemiplimab, nivolumab, or atezolizumab. In some embodiments, the combination comprises at least one HER2-targeted STING agonist antibody-drug conjugate of the disclosure and dostarlimab. In some embodiments, the combination comprises at least one HER2-targeted STING agonist antibody-drug conjugate of the disclosure and pembrolizumab.

In some embodiments, the combination comprising at least one HER2-targeted STING agonist antibody-drug conjugate of the disclosure is administered in combination with at least one immune checkpoint inhibitor.

In some embodiments, the combination comprising at least one HER2-targeted STING agonist antibody-drug conjugate of the disclosure is administered in combination with at least one PD-1 inhibitor or at least one PD-L1 inhibitor.

In some embodiments, the combination comprising at least one HER2-targeted STING agonist antibody-drug conjugate of the disclosure is administered in combination with avelumab, durvalumab, dostarlimab, pembrolizumab, cemiplimab, nivolumab, or atezolizumab. In some embodiments, the combination comprising at least one HER2-targeted STING agonist antibody-drug conjugate of the disclosure is administered in combination with dostarlimab. In some embodiments, the combination comprising at least one HER2-targeted STING agonist antibody-drug conjugate of the disclosure is administered in combination with pembrolizumab.

In some embodiments, the HER2-targeted STING agonist antibody-drug conjugate enhances the efficacy of the immunotherapy (e.g., an immune checkpoint inhibitor).

### Methods of Use

In some embodiments, the present disclosure provides a method of treating or preventing a disease or disorder in a subject in need thereof, comprising administering to the subject a therapeutically effective amount of at least one HER2-targeted STING agonist antibody-drug conjugate and at least one HER2-targeted therapy or at least one immune checkpoint inhibitor.

In some embodiments, the present disclosure provides a method of treating a disease or disorder in a subject in need thereof, comprising administering to the subject a therapeutically effective amount of at least one HER2-targeted STING agonist antibody-drug conjugate and at least one HER2-targeted therapy or at least one immune checkpoint inhibitor.

In some embodiments, the present disclosure provides a method of treating or preventing a disease or disorder in a subject in need thereof, comprising administering to the subject at least one HER2-targeted STING agonist antibody-drug conjugate and at least one HER2-targeted therapy or at least one immune checkpoint inhibitor.

In some embodiments, the present disclosure provides a method of treating a disease or disorder in a subject in need thereof, comprising administering to the subject at least one HER2-targeted STING agonist antibody-drug conjugate and at least one HER2-targeted therapy or at least one immune checkpoint inhibitor.

In some embodiments, the present disclosure provides a method of activating or enhancing an activity of STING in a subject, comprising administering to the subject a combination therapy disclosed herein i.e. at least one HER2-targeted STING agonist antibody-drug conjugate and at least one HER2-targeted therapy or at least one immune checkpoint inhibitor.

In some embodiments, the present disclosure relates to a method of treating a cancer in a subject in need thereof, comprising administering to the subject an effective amount of a combination therapy disclosed herein.

In some embodiments, the present disclosure relates to a method of treating a cancer in a subject in need thereof, comprising administering to the subject a combination therapy disclosed herein.

In some embodiments, the present disclosure provides a combination therapy disclosed herein for use in treating or preventing a disease or disorder in a subject in need thereof.

In some embodiments, the present disclosure provides a combination therapy disclosed herein for use in treating a disease or disorder in a subject in need thereof.

In some embodiments, the present disclosure provides a combination therapy disclosed herein for treating a STING-mediated disease or disorder in a subject.

In some embodiments, the present disclosure provides use of a combination therapy disclosed herein for treating or preventing a disease or disorder in a subject in need thereof.

In some embodiments, the present disclosure provides use of a combination therapy disclosed herein for treating a disease or disorder in a subject in need thereof.

In some embodiments, the present disclosure provides use of a combination therapy disclosed herein for treating a cancer in a subject in need thereof.

In some embodiments, the present disclosure provides use of a combination therapy disclosed herein for treating a STING-mediated disease or disorder in a subject in need thereof.

In some embodiments, the present disclosure provides use of a combination therapy disclosed herein in the manufacture of a medicament for treating a disease or disorder in a subject in need thereof.

In some embodiments, the present disclosure provides use of a combination therapy disclosed herein in the manufacture of a medicament for treating or preventing a disease or disorder in a subject in need thereof.

In some embodiments, the present disclosure provides use of a combination therapy disclosed herein in the manufacture of a medicament for treating a STING-mediated disease or disorder in a subject.

In some embodiments, the present disclosure provides use of a combination therapy disclosed herein in the manufacture of a medicament for treating a cancer in a subject in need thereof.

In some embodiments, the present disclosure provides use of a combination therapy disclosed herein for the treatment or prevention of a disease or disorder in a subject in need thereof.

In some embodiments, the present disclosure provides use of a combination therapy disclosed herein for the treatment of a disease or disorder in a subject in need thereof.

In some embodiments, the present disclosure provides use of a combination therapy disclosed herein for treating a STING-mediated disease or disorder in a subject.

In some embodiments, the present disclosure provides use of a combination therapy disclosed herein for treatment of a cancer in a subject in need thereof.

In some embodiments, the combination comprises at least one HER2-targeted STING agonist antibody-drug conjugate and at least one HER2-targeted therapy or at least one immune checkpoint inhibitor.

In some embodiments, the combination therapy disclosed herein is administered to the subject.

In some embodiments, the present disclosure provides a method of treating or preventing a disease or disorder in a subject in need thereof, comprising administering to the subject an efficient amount of at least one HER2-targeted STING agonist antibody-drug conjugate and at least one HER2-targeted therapy or at least one immune checkpoint inhibitor; wherein said HER2-targeted STING agonist antibody-drug conjugate releases one or more therapeutic agent upon biodegradation.

In some embodiments, the present disclosure provides a method of treating a disease or disorder in a subject in need thereof, comprising administering to the subject an efficient amount of at least one conjugate of the disclosure; wherein said HER2-targeted STING agonist antibody-drug conjugate releases one or more therapeutic agents upon biodegradation.

In some embodiments, the disease or disorder is a cancer.

In some embodiments, the cancer is selected from the group consisting of anal cancer, astrocytoma, leukemia, lymphoma, head and neck cancer, liver cancer, testicular cancer, cervical cancer, sarcoma, hemangioma, esophageal cancer, eye cancer, laryngeal cancer, mouth cancer, mesothelioma, skin cancer, myeloma, oral cancer, rectal cancer, colorectal cancer (CRC), throat cancer, bladder cancer, breast cancer, urothelial cancer, uterine cancer, ovarian cancer, prostate cancer, lung cancer, non-small cell lung cancer (NSCLC), colon cancer, pancreatic cancer, renal cancer, gastric cancer and gastric esophagogastric junction cancer.

In some embodiments, the cancer is selected from the group consisting of breast cancer, gastric cancer, gastric esophagogastric junction cancer, non-small cell lung cancer (NSCLC) or colorectal cancer.

In some embodiments, the combination therapies disclosed herein are useful in treating, preventing, delaying the progression of or otherwise ameliorating a symptom of breast cancer, gastric cancer, gastric esophagogastric junction cancer, non-small cell lung cancer (NSCLC) or colorectal cancer.

In some embodiments, the combination therapies disclosed herein are useful in treating, preventing, delaying the progression of or otherwise ameliorating a symptom of breast cancer. In some embodiments, the breast cancer is metastatic breast cancer. In some embodiments, the breast cancer is HER2 positive (HER2+) breast cancer. In some embodiments, the breast cancer is HER2 negative (HER2−) breast cancer. In some embodiments, the HER2+ breast cancer is metastatic HER2+ breast cancer. In some embodiments, the HER2− breast cancer is metastatic HER2− breast cancer.

In some embodiments, a subject having breast cancer has received at least one, at least two, at least three, or at least four prior lines of breast cancer therapy. In some aspects, a subject has received three or more prior lines of breast cancer therapy. In some aspects, a subject having HER2+ metastatic breast cancer has received three or more lines of breast cancer therapy.

In some embodiments, the combination therapies disclosed herein are useful in treating, preventing, delaying the progression of or otherwise ameliorating a symptom of gastric cancer.

In some embodiments, the combination therapies disclosed herein are useful in treating, preventing, delaying the progression of or otherwise ameliorating a symptom of gastric esophagogastric junction cancer.

In some embodiments, the combination therapies disclosed herein are useful in treating, preventing, delaying the progression of or otherwise ameliorating a symptom of colorectal cancer.

In some embodiments, the combination therapies disclosed herein are useful in treating, preventing, delaying the progression of or otherwise ameliorating a symptom of non-small cell lung cancer (NSCLC).

In some embodiments, the subject has recurrent or metastatic solid tumors with HER 2+ expression.

In some embodiments, the disease or disorder is a pre-cancerous syndrome.

In some embodiments, the combination therapies comprising at least one HER2-targeted STING agonist antibody-drug conjugate and at least one HER2-targeted therapy or at least one immune checkpoint inhibitor are useful in treating, preventing, delaying the progression of or otherwise ameliorating a symptom of breast cancer, gastric cancer, gastric esophagogastric junction cancer, non-small cell lung cancer (NSCLC) or colorectal cancer.

In some embodiments, the combination therapies comprising at least one HER2-targeted STING agonist antibody-drug conjugate and at least one HER2-targeted therapy are useful in treating, preventing, delaying the progression of or otherwise ameliorating a symptom of breast cancer, gastric cancer, gastric esophagogastric junction cancer, non-small cell lung cancer (NSCLC) or colorectal cancer.

In some embodiments, the combination therapies comprising at least one HER2-targeted STING agonist antibody-drug conjugate and at least one immune checkpoint inhibitor are useful in treating, preventing, delaying the progression of or otherwise ameliorating a symptom of breast cancer, gastric cancer, gastric esophagogastric junction cancer, non-small cell lung cancer (NSCLC) or colorectal cancer.

In some embodiments, the combination therapy comprising at least one HER2-targeted STING agonist antibody-drug conjugate is administered in combination with at least one HER2 antibody, at least one HER2 dimerization inhibitor antibody or a combination of at least one HER2 antibody and at least one HER2 dimerization inhibitor antibody for treating, preventing, delaying the progression of or otherwise ameliorating a symptom of breast cancer, gastric cancer, gastric esophagogastric junction cancer, non-small cell lung cancer (NSCLC) or colorectal cancer.

In some embodiments, the combination therapy comprising at least one HER2-targeted STING agonist antibody-drug conjugate and, is administered in combination with trastuzumab or pertuzumab or a combination thereof or margetuximab for treating, preventing, delaying the progression of or otherwise ameliorating a symptom of breast cancer, gastric cancer, gastric esophagogastric junction cancer, non-small cell lung cancer (NSCLC) or colorectal cancer.

In some embodiments, the combination therapy comprising at least one HER2-targeted STING agonist antibody-drug conjugate of the disclosure is administered in combination with at least one biosimilar of trastuzumab or at least one biosimilar of pertuzumab or a combination thereof or at least one biosimilar of margetuximab for treating, preventing, delaying the progression of or otherwise ameliorating a symptom of breast cancer, gastric cancer, gastric esophagogastric junction cancer, non-small cell lung cancer (NSCLC) or colorectal cancer.

In some embodiments, the combination therapy comprising at least one HER2-targeted STING agonist antibody-drug conjugate of the disclosure is administered in combination with at least one HER2-targeted antibody-drug conjugate, such as, for example, ado-trastuzumab emtansine (T-DM1) (Kadcyla®) or fam-trastuzumab deruxtecan (trastuzumab deruxtecan) (Enhertu®) for treating, preventing, delaying the progression of or otherwise ameliorating a symptom of breast cancer, gastric cancer, gastric esophagogastric junction cancer, non-small cell lung cancer (NSCLC) or colorectal cancer.

In some embodiments, the combination therapy comprising at least one HER2-targeted STING agonist antibody-drug conjugate of the disclosure is administered in combination with trastuzumab or a biosimilar of trastuzumab for treating, preventing, delaying the progression of or otherwise ameliorating a symptom of breast cancer, gastric cancer, gastric esophagogastric junction cancer, non-small cell lung cancer (NSCLC) or colorectal cancer. In some embodiments, the combination therapy comprising at least one HER2-targeted STING agonist antibody-drug conjugate of the disclosure is administered in combination with ado-trastuzumab emtansine (T-DM1) (Kadcyla®) or a biosimilar of ado-trastuzumab emtansine (T-DM1) (Kadcyla®) for treating, preventing, delaying the progression of or otherwise ameliorating a symptom of breast cancer, gastric cancer, gastric esophagogastric junction cancer, non-small cell lung cancer (NSCLC) or colorectal cancer. In some embodiments, the combination therapy comprising at least one HER2-targeted STING agonist antibody-drug conjugate of the disclosure is administered in combination with fam-trastuzumab deruxtecan (trastuzumab deruxtecan) (Enhertu®) or a biosimilar of fam-trastuzumab deruxtecan (trastuzumab deruxtecan) (Enhertu®) for treating, preventing, delaying the progression of or otherwise ameliorating a symptom of breast cancer, gastric cancer, gastric esophagogastric junction cancer, non-small cell lung cancer (NSCLC) or colorectal cancer.

In some embodiments, the combination therapy comprising at least one HER2-targeted STING agonist antibody-drug conjugate of the disclosure is administered in combination with trastuzumab or a biosimilar of trastuzumab for treating, preventing, delaying the progression of or otherwise ameliorating a symptom of HER2 positive (HER2+) metastatic breast cancer. In some embodiments, the combination therapy comprising at least one HER2-targeted STING agonist antibody-drug conjugate of the disclosure is administered in combination with trastuzumab or a biosimilar of trastuzumab for treating, preventing, delaying the progression of or otherwise ameliorating a symptom of HER2 positive (HER2+) metastatic breast cancer in a subject who has received at least one, at least two, at least three, or at least four prior lines of breast cancer therapy.

In some embodiments, the combination therapy comprising at least one HER2-targeted STING agonist antibody-drug conjugate of the disclosure is administered in combination with trastuzumab emtansine (T-DM1) (Kadcyla®) or a biosimilar of ado-trastuzumab emtansine (T-DM1) (Kadcyla®) for treating, preventing, delaying the progression of or otherwise ameliorating a symptom of HER2 positive (HER2+) metastatic breast cancer. In some embodiments, the combination therapy comprising at least one HER2-targeted STING agonist antibody-drug conjugate of the disclosure is administered in combination with trastuzumab emtansine (T-DM1) (Kadcyla®) or a biosimilar of ado-trastuzumab emtansine (T-DM1) (Kadcyla®) for treating, preventing, delaying the progression of or otherwise ameliorating a symptom of HER2 positive (HER2+) metastatic breast cancer in a subject who has received at least one, at least two, at least three, or at least four prior lines of breast cancer therapy.

In some embodiments, the combination therapy comprising at least one HER2-targeted STING agonist antibody-drug conjugate of the disclosure is administered in combination with fam-trastuzumab deruxtecan (trastuzumab deruxtecan) (Enhertu®) or a biosimilar of fam-trastuzumab deruxtecan (trastuzumab deruxtecan) (Enhertu®) for treating, preventing, delaying the progression of or otherwise ameliorating a symptom of HER2 positive (HER2+) metastatic breast cancer. In some embodiments, the combination therapy comprising at least one HER2-targeted STING agonist antibody-drug conjugate of the disclosure is administered in combination with fam-trastuzumab deruxtecan (trastuzumab deruxtecan) (Enhertu®) or a biosimilar of fam-trastuzumab deruxtecan (trastuzumab deruxtecan) (Enhertu®) for treating, preventing, delaying the progression of or otherwise ameliorating a symptom of HER2 positive (HER2+) metastatic breast cancer in a subject who has received at least one, at least two, at least three, or at least four prior lines of breast cancer therapy.

In some embodiments, the combination therapy comprising at least one HER2-targeted STING agonist antibody-drug conjugate of the disclosure is administered in combination with, at least one small molecule inhibitor of HER2, such as, for example, tucatinib, neratinib or lapatinib for treating, preventing, delaying the progression of or otherwise ameliorating a symptom of breast cancer, gastric cancer, gastric esophagogastric junction cancer, non-small cell lung cancer (NSCLC) or colorectal cancer.

In some embodiments, the composition therapy comprises at least one HER2-targeted STING agonist antibody-drug conjugate of the disclosure in combination with at least one immune checkpoint inhibitor are useful for treating, preventing, delaying the progression of or otherwise ameliorating a symptom of breast cancer, gastric cancer, gastric esophagogastric junction cancer, non-small cell lung cancer (NSCLC) or colorectal cancer.

In some embodiments, the composition therapy comprises at least one HER2-targeted STING agonist antibody-drug conjugate of the disclosure in combination with a monoclonal antibody, a humanized antibody, a fully human antibody, a fusion protein or a combination thereof are useful for treating, preventing, delaying the progression of or otherwise ameliorating a symptom of breast cancer, gastric cancer, gastric esophagogastric junction cancer, non-small cell lung cancer (NSCLC) or colorectal cancer.

In some embodiments, the combination therapy comprising at least one HER2-targeted STING agonist antibody-drug conjugate of the disclosure is administered in combination with at least one immune checkpoint inhibitor for treating, preventing, delaying the progression of or otherwise ameliorating a symptom of breast cancer, gastric cancer, gastric esophagogastric junction cancer, non-small cell lung cancer (NSCLC) or colorectal cancer.

In some embodiments, the combination therapy comprising at least one HER2-targeted STING agonist antibody-drug conjugate of the disclosure is administered in combination with at least one PD-1 inhibitor or at least one PD-L1 inhibitor for treating, preventing, delaying the progression of or otherwise ameliorating a symptom of breast cancer, gastric cancer, gastric esophagogastric junction cancer, non-small cell lung cancer (NSCLC) or colorectal cancer.

In some embodiments, the combination therapy comprising at least one HER2-targeted STING agonist antibody-drug conjugate of the disclosure is administered in combination with avelumab, durvalumab, dostarlimab, pembrolizumab, cemiplimab, nivolumab, or atezolizumab for treating, preventing, delaying the progression of or otherwise ameliorating a symptom of breast cancer, gastric cancer, gastric esophagogastric junction cancer, non-small cell lung cancer (NSCLC) or colorectal cancer. In some embodiments, the combination therapy comprising at least one HER2-targeted STING agonist antibody-drug conjugate of the disclosure is administered in combination with dostarlimab for treating, preventing, delaying the progression of or otherwise ameliorating a symptom of breast cancer, gastric cancer, gastric esophagogastric junction cancer, non-small cell lung cancer (NSCLC) or colorectal cancer. In some embodiments, the combination therapy comprising at least one HER2-targeted STING agonist antibody-drug conjugate of the disclosure is administered in combination with pembrolizumab for treating, preventing, delaying the progression of or otherwise ameliorating a symptom of breast cancer, gastric cancer, gastric esophagogastric junction cancer, non-small cell lung cancer (NSCLC) or colorectal cancer.

In some embodiments, the combination therapy comprising at least one HER2-targeted STING agonist antibody-drug conjugate of the disclosure is administered in combination with dostarlimab for treating, preventing, delaying the progression of or otherwise ameliorating a symptom of HER2 positive (HER2+) metastatic breast cancer. In some embodiments, the combination therapy comprising at least one HER2-targeted STING agonist antibody-drug conjugate of the disclosure is administered in combination with dostarlimab for treating, preventing, delaying the progression of or otherwise ameliorating a symptom of HER2 positive (HER2+) metastatic breast cancer in a subject who has received at least one, at least two, at least three, or at least four prior lines of breast cancer therapy.

In some embodiments, the combination therapy comprising at least one HER2-targeted STING agonist antibody-drug conjugate of the disclosure is administered in combination with pembrolizumab for treating, preventing, delaying the progression of or otherwise ameliorating a symptom of HER2 positive (HER2+) metastatic breast cancer. In some embodiments, the combination therapy comprising at least one HER2-targeted STING agonist antibody-drug conjugate of the disclosure is administered in combination with pembrolizumab for treating, preventing, delaying the progression of or otherwise ameliorating a symptom of HER2 positive (HER2+) metastatic breast cancer in a subject who has received at least one, at least two, at least three, or at least four prior lines of breast cancer therapy.

In some embodiment, the methods include identifying or otherwise refining, e.g., stratifying, a patient population suitable for therapeutic administration of the combination therapies thereof disclosed herein by identifying the HER2 score of subject by IHC test or FISH (fluorescence in situ hybridization negative) measurements prior to treatment with the combination therapies disclosed herein. The IHC test measures the amount of HER2 receptor protein on the surface of cells in a cancer tissue sample, e.g., a breast cancer tissue sample or a gastric cancer sample and assigns the detected level of cell surface HER2 receptor a HER2 score of 0, 1+, 2+ or 3+. If the subject's HER2 score is in the range of 0 to 1+, the cancer is deemed to be “HER2 negative.” If the score is 2+, the cancer is referred to as “borderline,” and a score of 3+ signifies that the cancer is “HER2 positive.”

In some embodiments, the subject is identified as having a scoring of 2+ or 3+ for HER2 expression as detected by immunohistochemistry (IHC) analysis performed on a test cell population, and the subject is also ER positive. In some embodiments, the subject is identified as having a scoring of 2+ or 3+ for HER2 expression as detected by immunohistochemistry (IHC) analysis performed on a test cell population, and the subject also is ER negative. In some embodiments, the subject is identified as having a scoring of 3+ for HER2 expression or evidence of gene amplification by FISH. In some embodiments, the subject is identified as having a scoring of 2+ for HER2 expression or evidence of gene amplification by FISH. In some embodiments, the subject is identified as having a scoring of 1+ for HER2 expression or evidence of gene amplification by FISH. In some embodiments, the subject is identified as having a high HER2 expression. In some embodiments, the subject is identified as having a low HER2 expression. In some embodiments, the test cell population is derived from fresh, unfrozen tissue from a biopsy sample. In some embodiments, the test cell population is derived from a frozen tissue from a biopsy sample.

In some embodiments, the combination therapy comprising at least one HER2-targeted STING agonist antibody-drug conjugate and at least one HER2-targeted therapy or at least one immune checkpoint inhibitor disclosed herein are useful in treating, preventing, delaying the progression of or otherwise ameliorating a symptom of breast cancer, gastric cancer, gastric esophagogastric junction cancer, colorectal cancer or non-small cell lung cancer (NSCLC) in patients who have HER2 IHC 2+ or HER2 IHC 3+. In some embodiments, the breast cancer patient is also ER positive. In some embodiments, the breast cancer patient is also ER negative.

In some embodiments, the combination therapy comprising at least one HER2-targeted STING agonist antibody-drug conjugate and at least one HER2-targeted therapy or at least one immune checkpoint inhibitor disclosed herein are useful in treating, preventing, delaying the progression of or otherwise ameliorating a symptom of breast cancer, gastric cancer, gastric esophagogastric junction cancer, colorectal cancer or non-small cell lung cancer (NSCLC) in patients who have HER2 IHC 1+ or HER2 IHC 2+.

In some embodiments, the combination comprising a HER2-targeted STING agonist antibody-drug conjugate and at least one HER2-targeted therapy or at least one immune checkpoint inhibitor disclosed herein are useful in treating, preventing, delaying the progression of or otherwise ameliorating a symptom of breast cancer, gastric cancer, gastric esophagogastric junction cancer, colorectal cancer or non-small cell lung cancer (NSCLC) in patients who have HER2 IHC 1+ or HER2 IHC 2+.

In some embodiments, the combination comprising a HER2-targeted STING agonist antibody-drug conjugate and at least one HER2-targeted therapy or at least one immune checkpoint inhibitor used in any of the embodiments of the methods and uses provided herein can be administered at any stage of the disease. For example, the combination therapy can be administered to a patient suffering cancer of any stage, from early to metastatic.

A combination therapy comprising at least one HER2-targeted STING agonist antibody-drug conjugate and at least one HER2-targeted therapy or at least one immune checkpoint inhibitor used in any of the embodiments of these methods and uses can be administered either without another therapeutic agent, or in further combination with one or more chemotherapeutic agents or other agents. In some embodiments, the additional agent is any of the toxins described herein. In some embodiments, the additional agent is (1) EGFR inhibitors (e.g., tyrosine kinase inhibitors or targeted anti-EGFR antibodies), (2) BRAF inhibitors, (3) ALK inhibitors, (4) hormone receptor inhibitors, (5) mTOR inhibitors, (6) VEGF inhibitors, or (7) cancer vaccines. In some embodiments, the additional agent is a standard, first line chemotherapeutic agent, such as, for example, ado-trastuzumab emtansine (Kadcyla), lapatinib, anastrozole, letrozole, exemestane, everolimus, fulvestrant, tamoxifen, toremifene, megestrol acetate, fluoxymesterone, ethinyl estradiol, paclitaxel, capecitabine, gemcitabine, eribulin, vinorelbine, cyclophosphamide, carboplatin, docetaxel, albumin-bound paclitaxel, cisplatin, epirubicin, ixabepilone, doxorubicin, fluorouracil, oxaliplatin, fluoropyrimidine, irinotecan, ramucirumab, mitomycin, leucovorin, cetuximab, bevacizumab, erlotinib, afatinib, crizotinib, permetrexed, ceritinib, etoposide, vinblastine, vincristine, ifosfamid, liposomal doxorubicin, topotecan, altretamine, melphalan or leuprolide acetate. In some embodiments, the additional agent is Kadcyla (ado-trastuzumab emtansine).

### Combination Therapies and Formulations

The combination therapies of the present disclosure may be administered together in a single pharmaceutical composition or separately and, when administered separately this may occur simultaneously or sequentially in any order. The amounts of the components of the combination of the present disclosure and the relative timings of administration will be selected in order to achieve the desired combined therapeutic effect.

The compositions of the disclosure may be administered once or according to a dosing regimen wherein a number of doses are administered at varying intervals of time for a given period of time. For example, doses may be administered daily, weekly, biweekly, monthly, bimonthly, every 6 months or annually. Doses may be administered until the desired therapeutic effect is achieved or indefinitely to maintain the desired therapeutic effect. Suitable dosing regimens for the compositions of the disclosure depend on the pharmacokinetic properties of that composition, such as absorption, distribution, and half-life, which can be determined by the skilled artisan. In addition, suitable dosing regimens, including the duration such regimens are administered, for a composition of the disclosure depend on the disease or disorder being treated, the severity of the disease or disorder being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

It will be appreciated that administration of the conjugates and HER2-targeted therapies or immune checkpoint inhibitors in the combinations of the disclosure will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed., Mack Publishing Company, Easton, Pa. (1975)), particularly Chapter 87 by Blaug, Seymour, therein.

For example, the combination therapy can include one or more conjugates disclosed herein co-formulated with, and/or co-administered with, one or more HER2-targeted therapy or one or more immune checkpoint inhibitor immune checkpoint inhibitors disclosed herein.

In some embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a conjugate disclosed herein) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration.

The pharmaceutical compositions are formulated to be compatible with its intended route of administration. Examples of routes of administration include parenteral, e.g., intravenous, intradermal, subcutaneous, oral (e.g., inhalation), transdermal (i.e., topical), transmucosal, and rectal administration.

In some embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a conjugate disclosed herein) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration.

For example, the combination therapy can include one or more conjugates disclosed herein co-formulated with, and/or co-administered with, one or more HER2-targeted therapies or one or more immune checkpoint inhibitors disclosed herein.

In some embodiments, the combinations comprising HER2-targeted STING agonist antibody-drug conjugates and HER2-targeted therapies or immune checkpoint inhibitors and additional agent(s) are formulated into a single therapeutic composition, and the components are administered simultaneously. Alternatively, the HER2-targeted STING agonist antibody-drug conjugate, HER2-targeted therapy or immune checkpoint inhibitor and additional agent, if any, are separate from each other, e.g., each is formulated into a separate therapeutic composition, and can be administered simultaneously, or at different times during a treatment regimen. For example, the HER2-targeted STING agonist antibody-drug is administered prior to the administration of the HER2-targeted therapy or immune checkpoint inhibitor; the HER2-targeted STING agonist antibody-drug is administered after the administration of the HER2-targeted therapy or immune checkpoint inhibitor. As described herein, the HER2-targeted STING agonist antibody-drug and the HER2-targeted therapy or the immune checkpoint inhibitor combination are administered in single doses or in multiple doses.

Dosage and Administration

The combination therapy provided herein, comprising a HER2-targeted STING agonist antibody-drug and a HER2-targeted therapy or an immune checkpoint inhibitor combination administered in single doses or in multiple doses is administered in an amount sufficient to exert a therapeutically useful effect. In some embodiments, the active agents are administered in an amount that does not result in undesirable side effects of the patient being treated, or that minimizes or reduces the observed side effects as compared to dosages and amounts required for single treatment with one of the above agents. For example, the combination therapy comprising a HER2-targeted STING agonist antibody-drug and a HER2-targeted therapy or the immune checkpoint inhibitor combination are administered in single doses or in multiple doses. Thus, it is possible, the amount of a HER2-targeted therapy or an immune checkpoint inhibitor that can be administered in the combination therapy provided herein, compared to the amount of the HER2-targeted therapy or the immune checkpoint inhibitor administered alone or using a known method is reduced, while achieving substantially the same or improved therapeutic efficacy. By virtue of the decreased dosage that can be administered, side effects associated with the HER2-targeted therapy or the immune checkpoint protein antibody administration, such as immune-related adverse events, described elsewhere or herein, are reduced, minimized or avoided.

It is within the level of one of skill in the art to determine the precise amounts of active agents, including HER2-targeted STING agonist antibody-drug conjugates and HER2-targeted therapies or immune checkpoint inhibitors to be administered to a subject. The dosages of such agents in a combination therapy can be chosen based on standard dosing regimens for that agent under a given route of administration.

It is understood that the precise dosage and duration of treatment is a function of the tissue or tumor being treated and may be determined empirically using known testing protocols or by extrapolation from in vivo or in vitro test data and/or can be determined from known dosing regimens of the particular agent. It is to be noted that concentrations and dosage values may also vary with the age of the individual treated, the weight of the individual, the route of administration and/or the extent or severity of the disease and other factors that are within the level of a skilled medical practitioner to consider. Generally, dosage regimens are chosen to limit toxicity. It should be noted that the treating physician would know how to and when to terminate, interrupt or adjust therapy to lower dosage due to toxicity, or bone marrow, liver or kidney or other tissue dysfunctions. Conversely, the treating physician would also know how to and when to adjust treatment to higher levels if the clinical response is not adequate (precluding toxic side effects). It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope thereof.

In some embodiments, the HER2-targeted STING agonist antibody-drug conjugate combination is administered in a therapeutically effective amount to decrease the tumor volume.

The amount of a HER2-targeted STING agonist antibody-drug conjugate administered for the treatment of a disease or condition, for example a cancer or solid tumor can be determined by standard clinical techniques. In addition, in vitro assays and animal models can be employed to help identify optimal dosage ranges. The precise dosage, which can be determined empirically, can depend on the route of administration, the type of disease to be treated and the seriousness of the disease.

The HER2-targeted therapy or the immune checkpoint inhibitor can be provided in a therapeutically effective amount for the particular dosage regimen. Therapeutically effective concentrations can be determined empirically by testing the compounds in known in vitro and in vivo systems, such as the assays provided herein. The concentration of a selected HER2-targeted therapy to immune checkpoint inhibitor in the composition depends on absorption, inactivation and excretion rates of the complex, the physicochemical characteristics of the complex, the dosage schedule, and amount administered as well as other factors known to those of skill in the art.

The amount of a selected HER2-targeted therapy or immune checkpoint inhibitor to be administered for the treatment of cancers can be determined by standard clinical techniques or other methods as described herein. In addition, in vitro assays and animal models can be employed to help identify optimal dosage ranges. Hence, the precise dosage, which can be determined empirically, can depend on route of administration, the type of cancer to be treated and the progression of the disease. If necessary, a particular dosage and duration and treatment protocol can be empirically determined or extrapolated. Dosage levels can be determined based on a variety of factors, such as body weight of the individual, general health, age, the activity of the specific compound employed, sex, diet, time of administration, rate of excretion, drug combination, the severity and course of the disease, and the patient's disposition to the disease and the judgment of the treating physician.

The combinations of the disclosure may also be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is, for example, by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin.

In some embodiments, the HER2-targeted STING agonist antibody-drug conjugates and HER2-targeted therapies or immune checkpoint inhibitors are administered as an infusion every one week, every two weeks, every three weeks, every four weeks, every five weeks, every six weeks, every seven weeks, or every eight weeks.

In some embodiments, the HER2-targeted STING agonist antibody-drug conjugates and HER2-targeted therapy or immune checkpoint inhibitor is administered as an infusion every three weeks or every four weeks.

In some embodiments, the HER2-targeted STING agonist antibody-drug and the HER2-targeted therapy or immune checkpoint inhibitor are administered by infusion simultaneously. In some embodiments, the HER2-targeted STING agonist antibody-drug is administered by infusion prior to the administration of the HER2-targeted therapy or immune checkpoint inhibitor. In some embodiments, the HER2-targeted STING agonist antibody-drug is administered by infusion after the administration of the HER2-targeted therapy or immune checkpoint inhibitor. As described herein, the HER2-targeted STING agonist antibody-drug and the HER2-targeted therapy or the immune checkpoint inhibitor combination can be administered in single doses or in multiple doses.

The frequency and timing of administration, and the dosage amounts, can be administered periodically over a cycle of administration to maintain a continuous and/or long term effect of the active agents for a desired length of time. The provided combination can be administered hourly, daily, weekly, monthly, yearly or once. The length of time of the cycle of administration can be empirically determined, and is dependent on the disease to be treated, the severity of the disease, the particular patient, and other considerations within the level of skill of the treating physician. The length of time of treatment with a combination therapy provided herein can be one week, two weeks, one months, several months, one year, several years or more.

In some embodiments, exemplary doses of intravenously administered immune checkpoint inhibitor, such as an anti-immune checkpoint protein antibody, can be used as a starting point to determine appropriate dosages. Dosage levels can be determined based on a variety of factors, such as body weight of the individual, general health, age, the activity of the specific compound employed, sex, diet, time of administration, rate of excretion, drug combination, the severity and course of the disease, and the patient's disposition resulting from the disease and the judgment of the treating physician.

It is understood that the amount to administer will be a function of the type of cancer being treated, the route of administration, and the tolerability of possible side effects. If necessary, dosage can be empirically determined.

For intravenous administration, one or more, or all, of the agents used in the combination therapy can be administered by push or bolus, by infusion, or via a combination thereof. The infusion time can be about 1 minute to three hours, such as about 1 minute to about two hours, about 1 minute to about 60 minutes, about 1 minute to about 90 minutes, or about 1 minute to about 120 minutes. The agents can be administered by concurrent infusion or by subsequent infusion. For example, the administered agents are administered separately and are provided in separate bags for separate infusions. In some embodiments, the HER2-targeted antibody-drug conjugate composition and the HER-2 targeted therapy or the immune checkpoint inhibitor composition are formulated and administered separately.

The HER2-targeted STING agonist antibody-drug conjugate can be administered prior to, simultaneously with or near simultaneously with, sequentially with or intermittently with the HER2-targeted therapy or the immune checkpoint inhibitor. For example, the HER2-targeted antibody-drug conjugate and the HER2-targeted therapy or the immune checkpoint inhibitor, e.g., an anti-immune checkpoint protein antibody (e.g., an anti-CTLA4 or anti-PD-1 antibody) can be co-administered or separately.

In some embodiments, the HER2-targeted STING agonist antibody-drug conjugate is administered prior to the HER2-targeted therapy or the immune checkpoint inhibitor. For example, the HER2-targeted STING agonist antibody-drug conjugate is administered up to about 48 hours prior to administering the HER2-targeted therapy or the immune checkpoint inhibitor. For example, the HER2-targeted STING agonist antibody-drug conjugate is administered about 5 minutes, 15 minutes, 30 minutes, 1 hour, 2 hours, 3 hours, 4 hours, 6 hours, 8 hours, 12 hours, 16 hours, 18 hours, 20 hours, 22 hours, 24 hours, 30 hours, 36 hours, 40 hours, or up to about 48 hours prior to administration of the HER2-targeted therapy or the immune checkpoint inhibitor.

In some embodiments, the HER2-targeted STING agonist antibody-drug conjugate is administered after the HER2-targeted therapy or the immune checkpoint inhibitor. For example, the HER2-targeted STING agonist antibody-drug conjugate is administered up to about 48 hours after administering the HER2-targeted therapy or the immune checkpoint inhibitor. For example, the HER2-targeted STING agonist antibody-drug conjugate is administered about 5 minutes, 15 minutes, 30 minutes, 1 hour, 2 hours, 3 hours, 4 hours, 6 hours, 8 hours, 12 hours, 16 hours, 18 hours, 20 hours, 22 hours, 24 hours, 30 hours, 36 hours, 40 hours, or up to about 48 hours after administration of the HER2-targeted therapy or the immune checkpoint inhibitor.

The frequency and timing of administration, and the dosage amounts, can be administered periodically over a cycle of administration to maintain a continuous and/or long-term effect of the active agents for a desired length of time and need not be the same for the HER2-targeted STING agonist antibody-drug conjugate and the HER2-targeted therapy or the immune checkpoint inhibitor. The provided compositions of each active agent or combinations thereof can be administered hourly, daily, weekly, monthly, yearly or once. The length of time of the cycle of administration can be empirically determined, and is dependent on the disease to be treated, the severity of the disease, the disposition of the patient, and other considerations within the level of skill of the treating physician. The length of time of treatment with a combination therapy provided herein can be one week, two weeks, one months, several months, one year, several years or more.

For example, the frequency of administration of the HER2-targeted STING agonist antibody-drug conjugate is once a day, every other day, twice weekly, once weekly, once every 2 weeks, once every 3 weeks or once every 4 weeks. The dosages can be divided into a plurality of cycles of administration during the course of treatment. For example, the HER2-targeted STING agonist antibody-drug conjugate can be administered at the frequency over a period of about a month, 2 months, 3 months, 4 months, 5 months, 6 months, a year or more. The frequency of administration can be the same throughout the period of the cycle or can differ. For example, an exemplary dosage frequency is two times a week at least for a first week of a cycle of administration. After the first week, the frequency can continue at twice a week, can increase to more than twice a week, or can be reduced to no more than once a week. It is within the level of a skilled person to determine the particular dosage frequency and cycle of administration based on the particular dosage being administered, the disease or condition being treated, the severity of the disease or condition, the age of the subject and other similar factors.

The HER2-targeted therapy or the immune checkpoint inhibitor can be administered at the same frequency or at a different frequency. For example, each administration of the HER2-targeted therapy or the immune checkpoint inhibitor is preceded by an administration of the HER2-targeted STING agonist antibody-drug conjugate by not more than 48 hours. For example, each dose of the HER2-targeted STING agonist antibody-drug conjugate is followed 24 to 48 hr later by a dose of the HER2-targeted therapy or the immune checkpoint inhibitor. In certain embodiments, the HER2-targeted therapy or the immune checkpoint inhibitor is administered less frequently than the HER2-targeted STING agonist antibody-drug conjugate, but each dose of the HER2-targeted therapy or the immune checkpoint inhibitor is preceded by a dose of the HER2-targeted antibody-drug conjugate. For example, the HER2-targeted therapy or the immune checkpoint inhibitor is administered twice weekly, once weekly, once every 2 weeks, once every 3 weeks, once every 4 weeks, once every 6 weeks, once every 2 months, once every 3 months, once every 4 months, once every 5 months, or once every 6 months, and in a manner that is preceded by administration of a HER2-targeted STING agonist antibody-drug conjugate. In another example, each dose of the HER2-targeted STING agonist antibody-drug conjugate is preceded by a dose of the HER2-targeted therapy or the immune checkpoint inhibitor. In certain embodiments, the HER2-targeted therapy or the immune checkpoint inhibitor is administered more frequently than the HER2-targeted STING agonist antibody-drug conjugate. For example, the HER2-targeted therapy or the immune checkpoint inhibitor is administered twice weekly, once weekly, once every 2 weeks, once every 3 weeks, once every 4 weeks, once every 6 weeks, once every 2 months, once every 3 months, once every 4 months, once every 5 months, or once every 6 months, and in a manner that some but not all HER2-targeted therapy dosages or checkpoint inhibitor dosages are followed by administration of a HER2-targeted STING agonist antibody-drug conjugate.

If disease symptoms persist in the absence of discontinued treatment, treatment can be continued for an additional length of time. Over the course of treatment, evidence of disease and/or treatment-related toxicity or side effects can be monitored.

The cycle of administration of the HER2-targeted STING agonist antibody-drug conjugate and/or HER2-targeted therapy and/or immune checkpoint inhibitor can be tailored to add periods of discontinued treatment in order to provide a rest period from exposure to the agents. The length of time for the discontinuation of treatment can be for a predetermined time or can be empirically determined depending on how the patient is responding or depending on observed side effects. For example, the treatment can be discontinued for one week, two weeks, one month or several months. Generally, the period of discontinued treatment is built into a cycle of dosing regimen for a patient.

An exemplary dosing regimen is a treatment cycle or cycle of administration of 21 or 28 days. The agent, such as the HER2-targeted STING agonist antibody-drug conjugate disclosed herein, can be administered on day 1, followed by administration of a HER2-targeted therapy or an immune checkpoint inhibitor of the disclosure, such as a HER2-targeted therapy or an immune checkpoint protein antibody on day 2, followed by 19 or 26 days without dosing. It is understood that the above description is for exemplification purposes only and that variations of the above can be employed. Further, similar cycles of administration can be applied to all administered agents, or each administered agent can be employed in its own dosing regimen in the combination therapy provided herein.

It is within the level of one of skill in the art to determine the precise cycle of administration and dosing schedule. As noted above, the cycle of administration can be for any desired length of time. Hence, the 21-day or 28-day cycle of administration can be repeated for any length of time. It is within the level of skill of the treating physician to adopt a cycle of administration and dosing regimen that meets the needs of the patient depending on personal considerations specific to the patient and disease to be treated.

### Diagnostic and Prophylactic Formulations

The conjugates and HER2-targeted therapy or immune checkpoint inhibitors disclosed herein are used in diagnostic and prophylactic formulations. In one embodiment, a HER2-targeted STING agonist antibody-drug conjugate and an HER2-targeted therapy or an immune checkpoint inhibitor disclosed herein are administered to patients that are at risk of developing one or more of the aforementioned diseases, such as for example, without limitation, cancer. A patient's or organ's predisposition to one or more of the aforementioned indications can be determined using genotypic, serological or biochemical markers.

In some embodiments, a HER2-targeted STING agonist antibody-drug conjugate and an immune checkpoint inhibitor disclosed herein are administered to human individuals diagnosed with a clinical indication associated with one or more of the aforementioned diseases, such as for example, without limitation, cancer. Upon diagnosis, a HER2-targeted STING agonist antibody-drug conjugate and a HER2-targeted therapy or an immune checkpoint inhibitor disclosed herein are administered to mitigate or reverse the effects of the clinical indication associated with one or more of the aforementioned diseases.

In some embodiments, a method for identifying a breast cancer patient amenable to treatment with the combinations of conjugates and HER2-targeted therapy or immune checkpoint inhibitors disclosed herein, comprise measuring the status of certain characteristics in a tumor sample obtained from the patient, and identifying the patient for treatment based on the status of certain characteristics in the tumor sample.

Antibodies disclosed herein are also useful in the detection of HER2 in patient samples and accordingly are useful as diagnostics. For example, HER2 antibody disclosed herein are used in in vitro assays, e.g., ELISA, to detect HER2 levels in a patient sample.

In some embodiments, a HER2 antibody disclosed herein is immobilized on a solid support (e.g., the well(s) of a microtiter plate). The immobilized antibody serves as a capture antibody for any HER2 that may be present in a test sample. Prior to contacting the immobilized antibody with a patient sample, the solid support is rinsed and treated with a blocking agent such as milk protein or albumin to prevent nonspecific adsorption of the analyte.

Subsequently the wells are treated with a test sample suspected of containing the antigen, or with a solution containing a standard amount of the antigen. Such a sample is, e.g., a serum sample from a subject suspected of having levels of circulating antigen considered to be diagnostic of a pathology. After rinsing away the test sample or standard, the solid support is treated with a second antibody that is detectably labeled. The labeled second antibody serves as a detecting antibody. The level of detectable label is measured, and the concentration of HER2 antigen in the test sample is determined by comparison with a standard curve developed from the standard samples.

It will be appreciated that based on the results obtained using the HER2 antibody disclosed herein in an in vitro diagnostic assay, it is possible to stage a disease in a subject based on expression levels of the HER2 antigen. For a given disease, samples of blood are taken from subjects diagnosed as being at various stages in the progression of the disease, and/or at various points in the therapeutic treatment of the disease. Using a population of samples that provides statistically significant results for each stage of progression or therapy, a range of concentrations of the antigen that may be considered characteristic of each stage is designated.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

The following examples illustrate the disclosure. These examples are not intended to limit the scope of the present disclosure, but rather to provide guidance to the skilled artisan to prepare and use the Compounds, compositions, and methods of the present disclosure. While particular embodiments of the present disclosure are described, the skilled artisan will appreciate that various changes and modifications can be made without departing from the spirit and scope of the disclosure.

It will be understood that certain Compounds of the disclosure may be potent immunomodulators and accordingly, care should be exercised in their handling.

Unless otherwise noted, all starting materials were obtained from commercial suppliers and used without further purification.

### Abbreviations

The following abbreviations are used in the reaction schemes and synthetic examples, which follow. This list is not meant to be an all-inclusive list of abbreviations used in the application as additional standard abbreviations, which are readily understood by those skilled in the art of organic synthesis, can also be used in the synthetic schemes and examples.

| | |
|---|---|
| CHT | Ceramic hydroxyapatite |
| CR | Complete response |
| HIC | Hydrophobic interaction chromatography |
| HPLC | High performance liquid chromatography |
| PR | Partial response |
| RP-HPLC | Reverse Phase high performance liquid chromatography |
| SEC | Size exclusion chromatography |
| TCEP | (tris(2-carboxyethyl)phosphine) |
| TGD | Tumor growth delay |
| TGI | Tumor growth inhibition |
| TFS | Tumor-free survivor |

### General Information

All reagents were purchased from relevant providers unless otherwise stated.

XMT-1519 (anti-Her2 antibody) is disclosed in U.S. Pat. No. 9,555,112, issued Jan. 31, 2017 and U.S. Pat. No. 9,738,720, issued Aug. 22, 2017, the entire contents of which are incorporated herein by reference.

The antibody-drug conjugate Enhertu is also known as trastuzumab deruxtecan or T-DXd.

7.16.4_msIgG2a mab is a murine IgG2a antibody that recognizes the rat neu oncogene-encoded p185 molecule (rat HER2(neu)), As the humanized antibody, XMT-1519, does not bind rat HER2, Conjugates 1 and 2 could not be used in the rat HER2 EMT6-RHER2-MSA mammary carcinoma tumor model. Hence surrogate Conjugates (7.16.4_msIgG2a Conjugate and non-binding control palivizumab_msIgG2a conjugate, Conjugate 4 and Conjugate 3 respectively) were used in the rat HER2 EMT6-RHER2-MSA mammary carcinoma tumor model as they specifically recognize rat HER2(neu).

HPLC purification was performed on a Phenomenex Gemini 5 μm C18 110 Å, 250×10 mm, semi-preparative column.

When applicable, the drug content of the conjugates was determined spectrophotometrically, otherwise RP-HPLC or LC/MS as performed for quantitative determination of the drug content.

The protein content of the antibody-drug conjugates was determined spectrophotometrically or by ELISA.

Antibody-drug conjugates, drug carrying Scaffolds, or antibody Scaffolds were purified (i.e., removal of residual unreacted drug, unconjugated antibody, enzymes or starting materials) by ultrafiltration, extensive diafiltration, CHT chromatography or HIC, as required. If necessary, additional purification by SEC or HIC were conducted to remove aggregated antibody-drug conjugates. In general, the antibody-drug conjugates, as purified, contained <5% (w/w) (e.g., <2% (w/w)) aggregated antibody-drug conjugates as determined by SEC; <0.5% (w/w) (e.g., <0.1% (w/w)) free (unconjugated) drug as determined by RP-HPLC and/or LC-MS/MS; <1% (w/w) of free drug conjugate as determined by SEC and/or RP-HPLC; and <10% (w/w) (e.g., <1% (w/w)) unconjugated antibody or antibody fragments as determined by HIC-HPLC and/or RP-HPLC. Reduced or partially reduced antibodies were prepared using procedures described in the literature, see, for example, Francisco et al., Blood 102 (4): 1458-1465 (2003). The total drug (conjugated and unconjugated) concentration was determined by UV-Vis spectrophotometry or RP-HPLC.

The drug to antibody ratio (DAR) was determined by measuring the absorption of the conjugates. The DAR value was calculated using the appropriate molar extinction coefficients of the antibody and the STING agonist payload.

Therapeutic agents, including HER2-targeted STING agonist ADCs, HER2-targeted therapies, and/or immunotherapies can be administered or dosed at the frequencies and intervals disclosed herein. In some aspects, therapeutic administration schedules can be as follows. Administration schedules for therapeutic agents

| | |
|---|---|
| QD | Single administration |
| QD X 1 | Single administration on day 1 |
| QWK | Administration once per week |
| QWK X 2 | Administration once per week for 2 weeks |
| QWK X 3 | Administration once per week for 3 weeks |
| BIW | Twice weekly administration |
| BIW X 2 | Twice weekly administration for 2 weeks |

Dosages of antibody/payload indicate mg of antibody per kilogram of body weight and mg of payload per kilogram of body weight. By way of example a conjugate dosage of 0.30/0.011 mg/kg indicates that 0.30 mg of the antibody is administered per kilogram of body weight and 0.011 mg of conjugate payload is administered per kilogram of body weight.

Tumors were measured twice weekly using digital calipers and tumor volumes were calculated using the formula: tumor volume (mm³)=(width²×length)/2. Body weights were recorded daily for the first week and twice weekly thereafter. Animals remained on study until individual tumor volume reached >1000 mm³, >1500 mm³ or as indicated. Percent change in body weight was calculated using the formula: body weight change (%)=((weight study day X−weight_{study day 1})/weight^{study day 1})*100. Tumor volumes are reported as mean±standard error of the mean (SEM). Tumor growth inhibition (% TGI) was defined as the percent difference in mean tumor volumes (MTVs) between treated and control groups. Tumor size was measured throughout each efficacy study to determine tumor growth inhibition (TGI). Percent tumor regression was calculated using the formula: % regression=(1−(mean tumor volume^{final})/(mean tumor volume^{day 1}))*100. A partial response (PR) is defined as a tumor volume of 50% or less for day 1 volume for three consecutive measurements and equal to or greater than 13.5 mm³ for at least one of these three measurements. A complete response (CR) is defined as a tumor volume less than 13.5 mm³ for three consecutive measurements. A tumor-free survivor (TFS) is classified as having a CR at the end of study.

### Example 1: Synthesis of XMT-1519 Conjugate 1, DAR 8.1

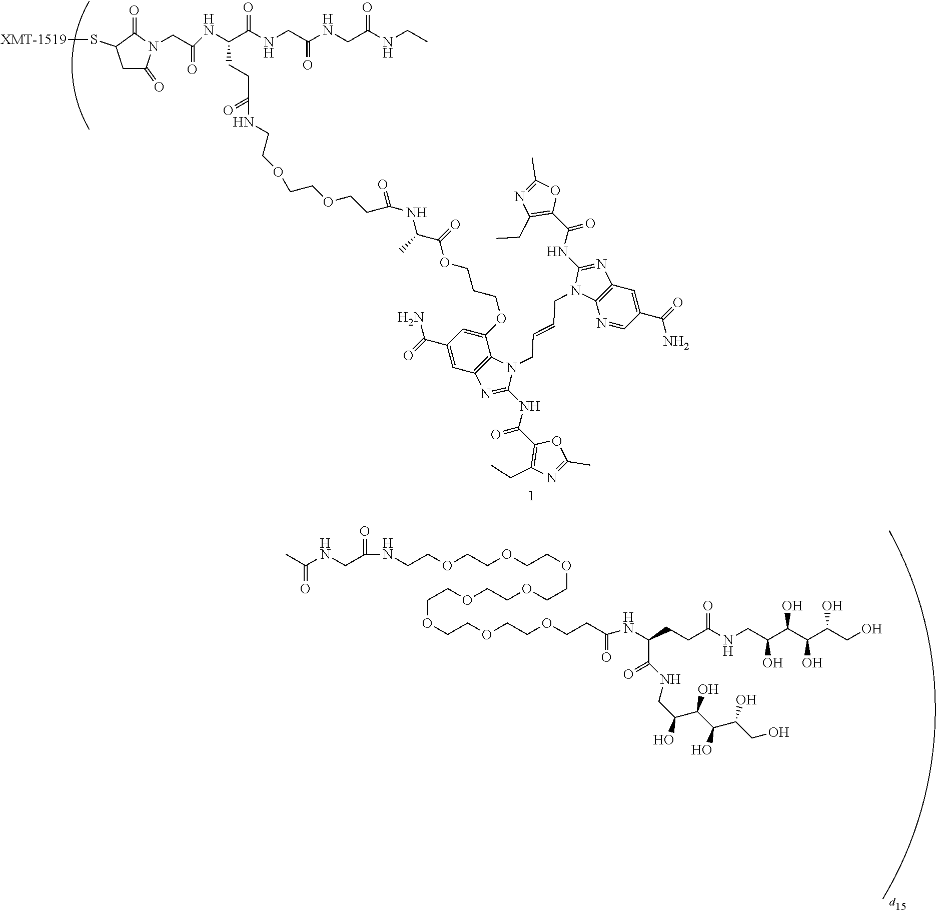

Conjugate 1 was prepared as described as described in co-pending application U.S. Ser. No. 17/221,341, filed Apr. 4, 2021. The purified Conjugate 1 had a STING agonist to XMT-1519 of 8.1.

### Example 2: Synthesis of Palivizumab Conjugate 2, DAR 6.8

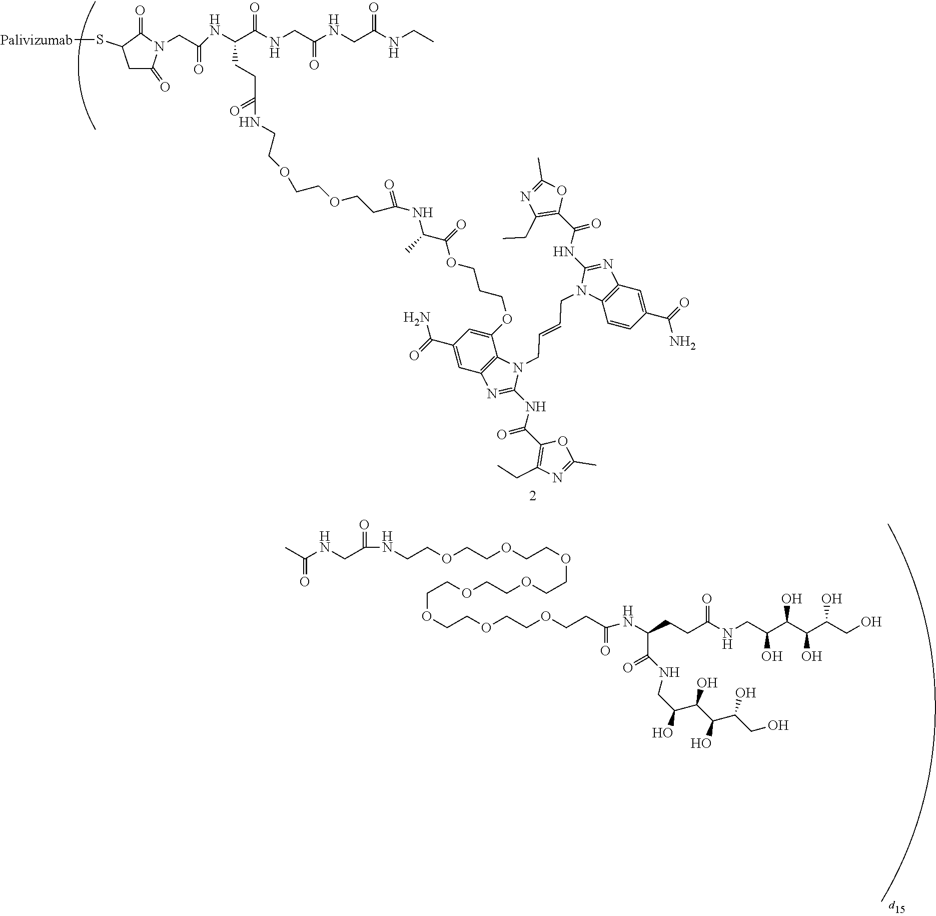

Conjugate 2 was prepared as described as described in co-pending application U.S. Ser. No. 17/221,341, filed Apr. 4, 2021. The purified Conjugate 2 had a STING agonist to Palivizumab ratio of 6.8.

### Example 3: Synthesis of Palivizumab_msIgG2a Conjugate 3, DAR 7.7

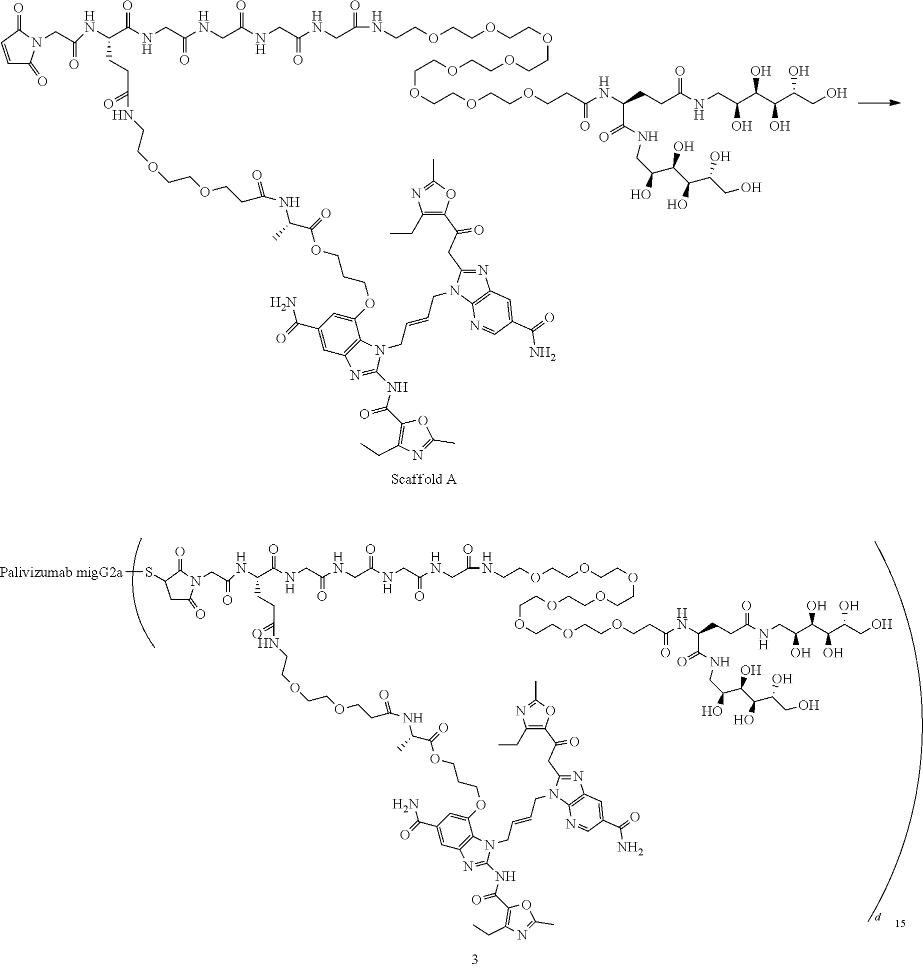

To a solution of Palivizumab_msIgG2a (90 mg, 0.617 μmol) in 50 mM HEPES, 1 mM EDTA, pH 7 buffer was added TCEP-HCl (1.76 mg, 6.17 μmol) with a final antibody concentration of 5 mg/mL and the mixture was shaken at 37° C. for 4 hours. To the reduced antibody was added Scaffold A (prepared as described in co-pending application U.S. Ser. No. 17/221,341, filed Apr. 4, 2021, 17.45 mg, 7.404 μmol in 1800 μL DMA). The resulting mixture was shaken at 37° C. for 120 minutes. The reaction was quenched with cysteine (15 equivalents, 1.121 mg, 9.255 μmol in 1.12 mL of 50 mM HEPES, 1 mM EDTA, pH 7) and rotated at room temperature for 1 h. The resulting Conjugate 3 was purified by ultrafiltration or CHT chromatography (80 mg, 90% yield). The purified Conjugate 3 had a STING agonist to Palivizumab_msIgG2a ratio of 7.7.

### Example 4: Synthesis of 7.16.4_msIgG2a Conjugate 4, DAR 6.9

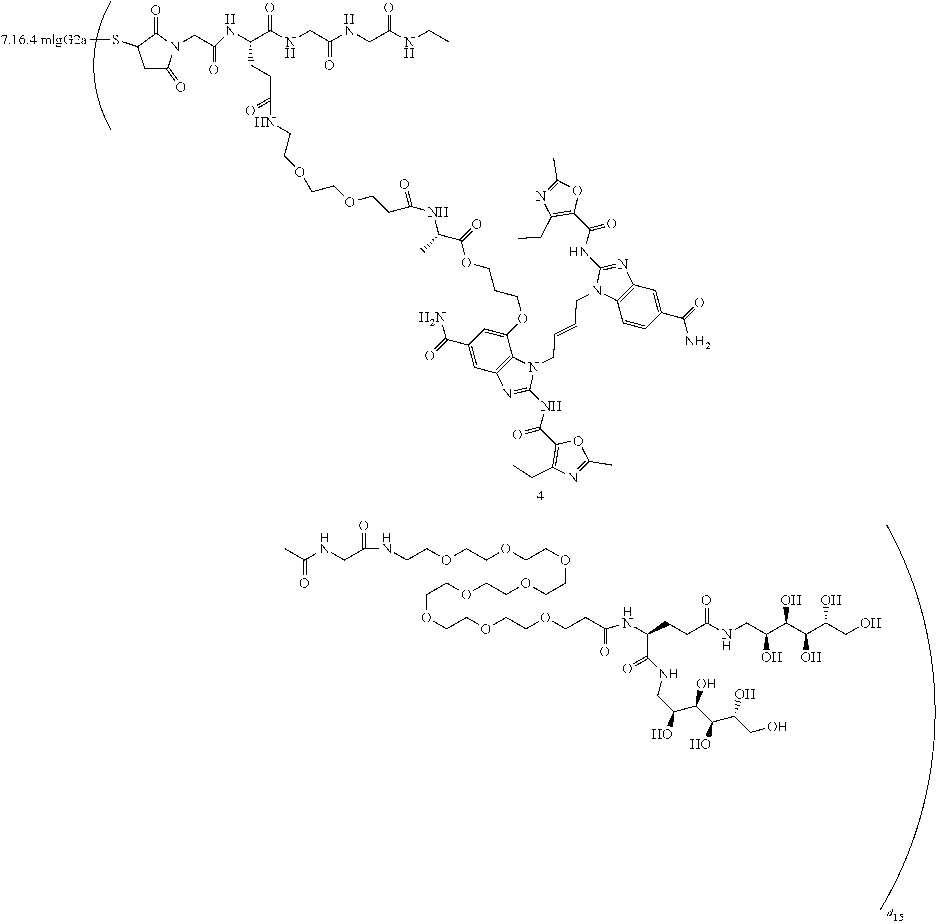

Conjugate 4 was prepared and characterized as described in Example 3 except that 7.16.4_msIgG2a was used instead of Palivizumab_msIgG2a. The purified Conjugate 4 had a STING agonist to 7.16.4_msIgG2a ratio of 6.9.

### Example 5: Tumor Growth Response to Administration of HER2 STING Agonist Antibody-Drug Conjugates in Combination with Trastuzumab in SKOV3 Ovarian Cancer

Female CB.17 SCID mice were inoculated subcutaneously with SKOV3 human ovarian cancer cells (10×10⁶ cells/mouse). SKOV3 cells are a breast cancer cell line having high HER2 expression. Animals were randomized into treatment groups when tumor volumes were between 60-100 mm³ (mean=72-81 mm³/group). The vehicle, Conjugate 1 (0.30/0.013 mg/kg), a combination of Conjugate 1 (0.30/0.013 mg/kg) and trastuzumab (3 mg/kg), a combination of Conjugate 1 (0.30/0.013 mg/kg) and trastuzumab (10 mg/kg), and a combination of Conjugate 2 (0.30/0.011 mg/kg) and trastuzumab (10 mg/kg), were dosed on qd×1 on day 1 or qwk×3 on days 1, 8 and 15. Additionally, trastuzumab (10 mg/kg), was dosed qwk×3 starting on day 1, 8 and 15. For all the doses, the Conjugates were dosed intravenously (IV); trastuzumab was dosed intraperitoneally (IP); all doses of the Conjugates are given as antibody/payload; n=10 for each group.

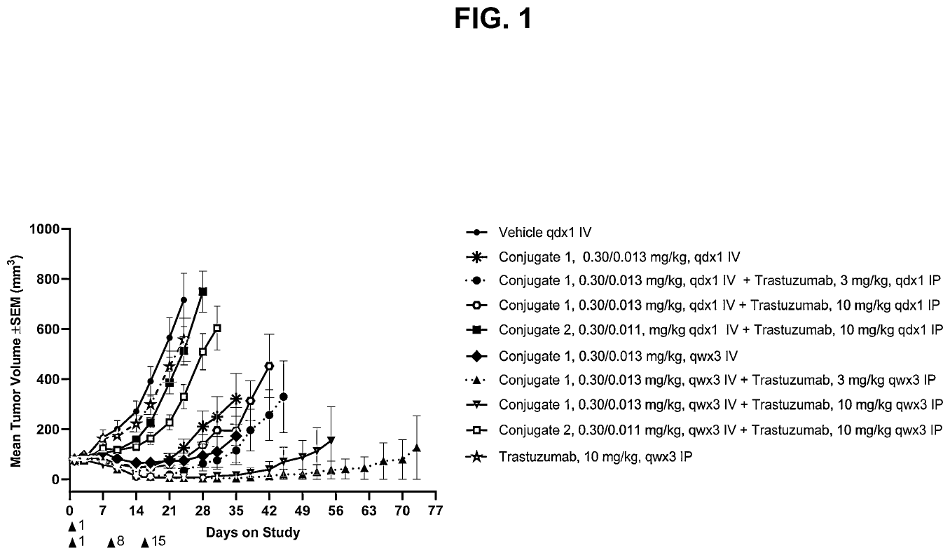

FIG. 1 provides the results for the tumor volumes of SKOV3 tumor-bearing mice treated with Vehicle, Conjugate 1, trastuzumab, a combination of Conjugate 2 and trastuzumab, and a combination of Conjugate 1 and trastuzumab at varying dose levels and dosing regimens. Treatment with Conjugate 1 (0.30/0.013 mg/kg, qd×1) resulted in 1 PR and 3 CR. Treatment with Conjugate 1 (0.30/0.013 mg/kg, qd×1) and trastuzumab (3 mg, qd×1) resulted in 7 CR and 3 TFS. Treatment with Conjugate 1 (0.30/0.013 mg/kg, qd×1) and trastuzumab (10 mg, qd×1) resulted in 2 CR and 1 TFS. Treatment with Conjugate 1 (0.30/0.013 mg/kg, qwk×3) resulted in 4 CR and 2 TFS. Treatment with Conjugate 1 (0.30/0.013 mg/kg, qwk×3) and trastuzumab (3 mg, qwk×3) resulted in 1 PR and 9 CR and 9 TFS. Treatment with Conjugate 1 (0.30/0.013 mg/kg, qwk×3) and trastuzumab (10 mg, qwk×3) resulted in 2 PR and 7 CR and 7 TFS. The results show that the addition of trastuzumab synergistically increases the efficacy of Conjugate 1.

### Example 6: Tumor Growth Response to Administration of HER2 STING Agonist Antibody-Drug Conjugates in Combination with Trastuzumab or Pertuzumab in JIMT-1

Female CB.17 SCID mice were inoculated subcutaneously with JIMT-1 tumor cells (10×10⁶ cells/mouse). JIMT-1 cells are a breast cancer cell line having moderate HER2 expression. Animals were randomized into treatment groups when tumor volumes were between 60-100 mm³ (mean 67.8-71.6 mm³/group). The Vehicle, Conjugate 1 (3.0/0.13 mg/kg), a combination of Conjugate 2 (3.0/0.11 mg/kg) and trastuzumab (10 mg/kg), a combination of Conjugate 2 (3.0/0.11 mg/kg) and pertuzumab (10 mg/kg), a combination of Conjugate 1 (3.0/0.13 mg/kg) and trastuzumab (3 mg/kg), a combination of Conjugate 1 (3.0/0.13 mg/kg) and trastuzumab (10 mg/kg), a combination of Conjugate 1 (3.0/0.13 mg/kg) and pertuzumab (3 mg/kg), a combination of Conjugate 1 (3.0/0.13 mg/kg) and pertuzumab (10 mg/kg), a combination of Conjugate 2 (3.0/0.11 mg/kg), trastuzumab (3 mg/kg) and pertuzumab (3 mg/kg), and a combination of Conjugate 1 (3.0/0.13 mg/kg), trastuzumab (3 mg/kg) and pertuzumab (3 mg/kg) were dosed on qd×1 on day 1. Additionally, Conjugate 1 (0.30/0.013 mg/kg), a combination of Conjugate 2 (0.30/0.011 mg/kg) and trastuzumab (3 mg/kg), a combination of Conjugate 2 (0.30/0.011 mg/kg) and pertuzumab (3 mg/kg), a combination of Conjugate 1 (0.30/0.013 mg/kg) and trastuzumab (3 mg/kg), and a combination of Conjugate 1 (0.30/0.013 mg/kg) and pertuzumab (3 mg/kg) were dosed qwk×3 on days 1, 8 and 15. For all the doses, the Conjugates were dosed intravenously; trastuzumab and pertuzumab were dosed intraperitoneally; all doses of the Conjugates are given as antibody/payload; n=10 for each group.

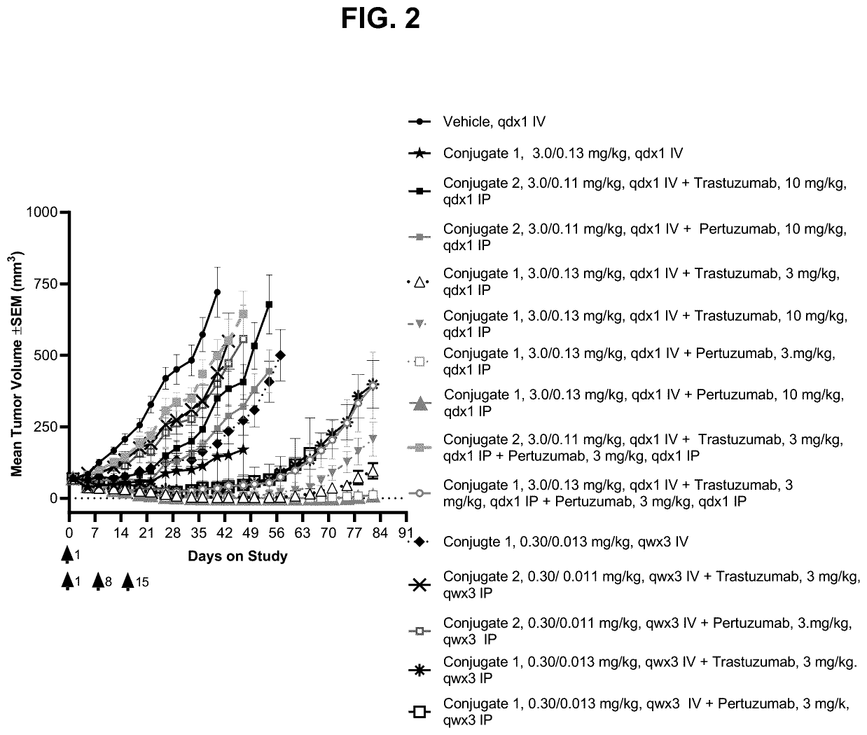

FIG. 2 provides the results for the tumor volumes of JIMT-1 tumor-bearing mice treated with Vehicle, Conjugate 1, a combination of Conjugate 1 and trastuzumab, a combination of Conjugate 1 and pertuzumab, a combination of Conjugate 1, trastuzumab, and pertuzumab, a combination of Conjugate 2 and trastuzumab, a combination of Conjugate 2 and pertuzumab, and a combination of Conjugate 2, trastuzumab, and pertuzumab at varying dose levels and dosing regimens.

All treatment groups showed significant tumor growth delay (TGD) compared to the vehicle control. However, only groups treated with Conjugate 1 (3 mg/kg) alone or in combination with trastuzumab and/or pertuzumab and Conjugate 1 (0.3 mg/kg) in combination with trastuzumab or pertuzumab achieved the maximum possible TGD (78%), although these groups differed in the number of regression responses and tumor free survivors (TFS) they produced. Treatment with Conjugate 1 (3 mg/kg, qd×1) and pertuzumab (3 or 10 mg/kg, qd×1) resulted in 9 TFS and 8 TFS, respectively, with 10 CRs each. Treatment with Conjugate 1 (3 mg/kg, qd×1) and trastuzumab (3 or 10 mg/kg, qd×1) resulted in 3 TFS each, with 10 CRs and 9 CRs and 1 PR respectively. All but one treatment group Conjugate 2, trastuzumab and pertuzumab showed significant TGI compared to the vehicle control.

### Example 7: Tumor Growth Response to Administration of HER2 STING Agonist Antibody-Drug Conjugates in Combination with Trastuzumab or Pertuzumab in SNU-5

Female CB.17 SCID mice were inoculated subcutaneously with SNU-5 tumor cells (10×10⁶ cells/mouse). SNU-5 are a gastric cancer cell line having low HER2 expression. Animals were randomized into treatment groups when tumor volumes were between 60-100 mm³ (mean 79.7-81.3 mm³/group). The Vehicle, Conjugate 1 (0.20/0.007 mg/kg), trastuzumab (2 mg/kg), pertuzumab (2 mg/kg), Conjugate 2 (0.20/0.007 mg/kg), a combination of Conjugate 1 (0.20/0.007 mg/kg) and trastuzumab (2 mg/kg), a combination of Conjugate 1 (0.20/0.007 mg/kg) and pertuzumab (2 mg/kg), a combination of trastuzumab (2 mg/kg) and pertuzumab (2 mg/kg), and a combination of Conjugate 1 (0.20/0.007 mg/kg), trastuzumab (2 mg/kg), and pertuzumab (2 mg/kg) were dosed on qd×1 on day 1, Additionally Conjugate 1 (0.10/0.004 mg/kg), Conjugate 2 (0.10/0.004 mg/kg), a combination of Conjugate 1 (0.10/0.004 mg/kg) and trastuzumab (2 mg/kg), and a combination of Conjugate 1 (0.10/0.004 mg/kg) and pertuzumab (2 mg/kg) for which the Conjugates were dosed qwk×3 on days 1, 8 and 15 and the trastuzumab or pertuzumab were dosed qd×1 on day 1. For all the doses, the Conjugates were dosed intravenously; trastuzumab and pertuzumab were dosed intraperitoneally; all doses of the Conjugates are given as antibody/payload; n=10 for each group).

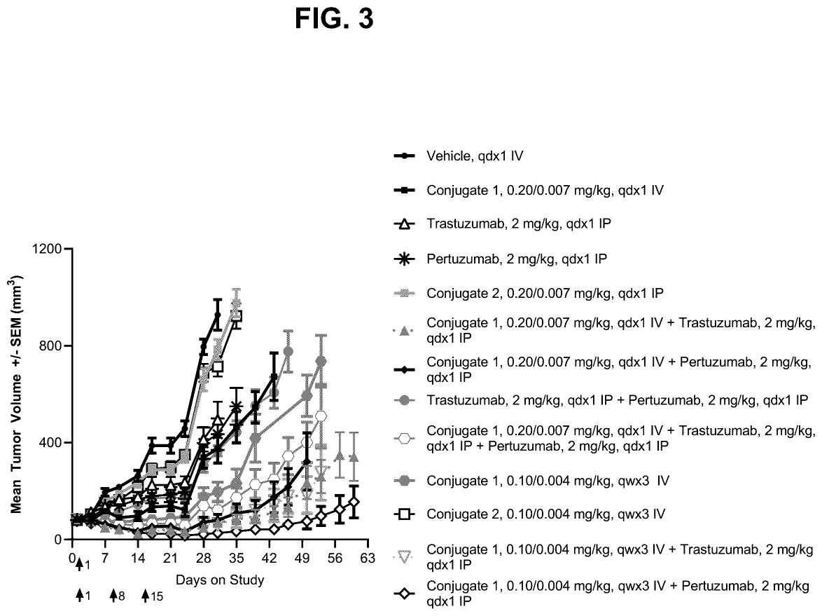

FIG. 3 provides the results for the tumor volumes of SNU-5 tumor-bearing mice treated with Vehicle, Conjugate 1, Conjugate 2, trastuzumab, pertuzumab, a combination of trastuzumab and pertuzumab, a combination of Conjugate 1 and trastuzumab, a combination of Conjugate 1 and pertuzumab, and a combination of Conjugate 1, trastuzumab, and pertuzumab at varying dose levels and dosing regimens.

### Example 8: Tumor Growth Response to Administration of HER2 STING Agonist Antibody-Drug Conjugates in Combination with Enhertu in JIMT-1

Female CB.17 SCID mice were inoculated subcutaneously with JIMT-1 tumor cells (10×10⁶ cells/mouse). Animals were randomized into treatment groups when tumor volumes were between 60-100 mm³ (mean 76.8 mm³/group). The Vehicle, Conjugate 2 (1.0/0.037 mg/kg), Conjugate 1 (1.0/0.043 mg/kg), Enhertu (1.0/0.026 mg/kg, 3.0/0.078 mg/kg or 10.0/0.261 mg/kg), a combination of Conjugate 1 (1.0/0.043 mg/kg) and Enhertu (1.0/0.026 mg/kg), a combination of Conjugate 1 (1.00/0.043 mg/kg) and Enhertu (3.0/0.078 mg/kg), and a combination of Conjugate 1 (1.00/0.043 mg/kg) and Enhertu (10.0/0.26 mg/kg) were dosed in which the Conjugates were dosed intravenously qd×1 on day 1 and Enhertu was dosed intravenously qwk×2 on days 1 and 8. Additionally, Conjugate 1 (0.30/0.013 mg/kg), a combination of Conjugate 1 (0.3/0.13 mg/kg) and Enhertu (1.0/0.026 mg/kg), a combination of Conjugate 1 (0.3/0.13 mg/kg) and Enhertu (3.0/0.078 mg/kg), and a combination of Conjugate 1 (0.3/0.13 mg/kg) and Enhertu (10.0/0.261 mg/kg) were dosed intravenously qwk×2 on days 1 and 8; all doses of the Conjugates are given as antibody/payload; n=10 for each group.

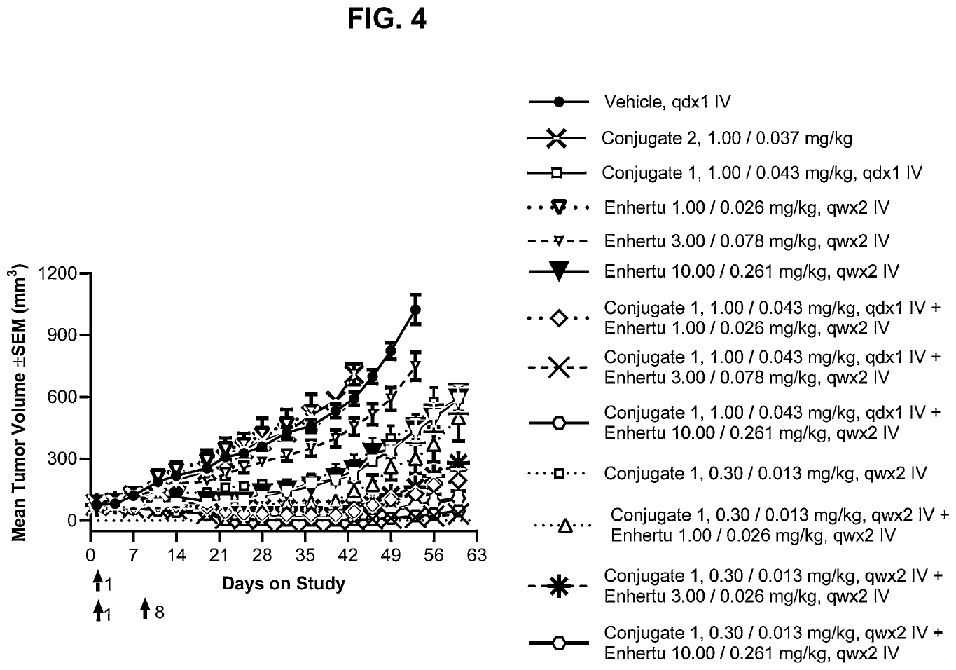

FIG. 4 provides the results for the tumor volumes of JIMT-1 tumor-bearing mice treated with Vehicle, Conjugate 1, Conjugate 2, Enhertu and a combination of Conjugate 1 and Enhertu at varying dose levels and dosing regimens.

### Example 9: Tumor Growth Response to Administration of HER2 STING Agonist Antibody-Drug Conjugates in Combination with Anti-PD-1 in Rat HER2 EMT6-RHER2-MSA Mammary Carcinoma

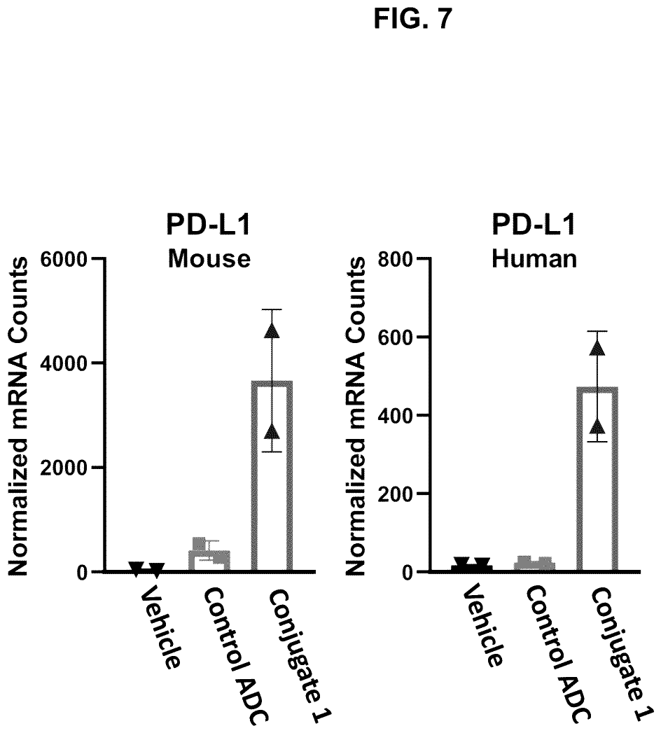

PD-L1 is upregulated in SKOV3 tumors after treatment with Conjugate 1 (FIG. 7), suggesting that combining conjugate 1 with an immune therapy such as a PD-1 checkpoint inhibitor may be efficacious. PD-L1 was observed to be upregulated in mice and human cells following treatment with Conjugate 1.

Female BALB/c mice were inoculated subcutaneously with EMT6-RHER2 MSA (engineered EMT6 cell line overexpressing rat epidermal growth factor receptor 2 (RHER2)) (5×10⁶ cells/mouse). Animals were randomized into treatment groups when tumor volumes were between 60-100 mm³ (mean=95-109 mm³/group). The Vehicle, Conjugate 3 (0.30/0.013 mg/kg, 1.0/0.04 mg/kg, or 3.0/0.12 mg/kg), Conjugate 4 (0.30/0.012 mg/kg, 1.0/0.035 mg/kg or 3.0/0.104 mg/kg) were all dosed on qd×1 on day 1. Anti-PD-1 RMP1-14 (1 mg/kg or 5 mg/kg) was dosed biw×2 on days 1, 4, 8 and 11. A combination of Conjugate 3 (0.30/0.012 mg/kg) and anti-PD-1 RMP1-14 (5 mg/kg), a combination of Conjugate 3 (I/O/0.035 mg/kg) and anti-PD-1 RMP1-14 (5 mg/kg), a combination of Conjugate 4 (0.30/0.012 mg/kg) and anti-PD-1 RMP1-14 (5 mg/kg), a combination of Conjugate 4 (1.0/0.035 mg/kg) and anti-PD-1 RMP1-14 (5 mg/kg) for which the Conjugates were dosed qd×1 on day 1 and anti-PD-1 RMP1-14 was dosed biw×2 on days 1, 4, 8 and 11. For all the doses the Conjugates were dosed intravenously; anti-PD-1 RMP1-14 was dosed intraperitoneally; all doses of the Conjugates are given as antibody/payload; n=10 for each group.

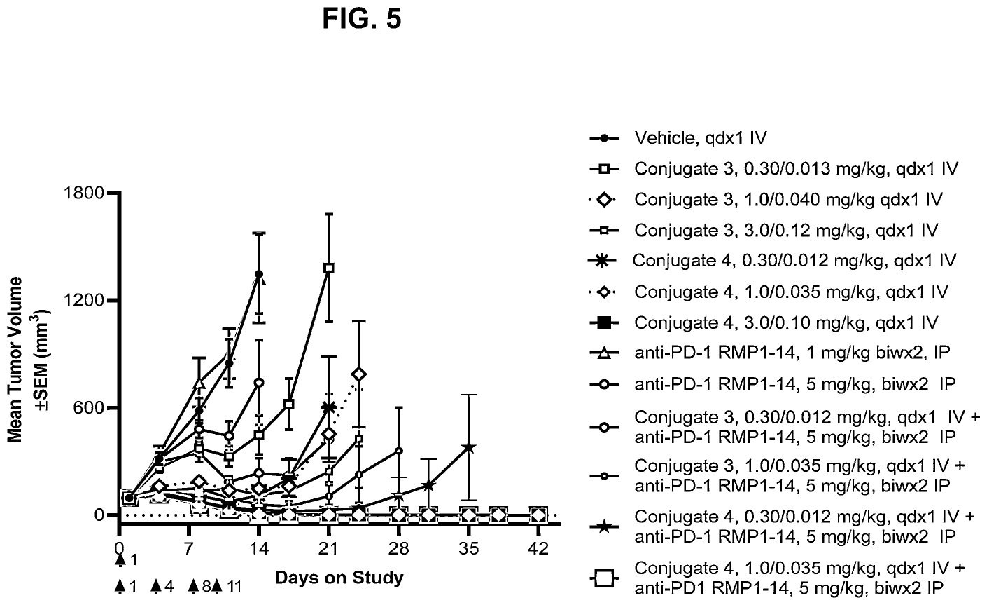

FIG. 5 provides the results for the tumor volumes of EMT6-RHER2 MSA tumor-bearing mice treated with Vehicle, Conjugate 3, Conjugate 4, anti-PD-1 RMP1-14, a combination of Conjugate 3 and anti-PD-1 RMP1-14, and a combination of Conjugate 4 and anti-PD-1 RMP1-14 at varying dose levels and dosing regimens. Treatment with Conjugate 3 (1.0/0.04 mg/kg) resulted in 5 CR and 5 TFS. Treatment with Conjugate 3 (3.0/0.12 mg/kg) resulted in 7 CR and 7 TFS. Treatment with Conjugate 4 (0.3/0.012 mg/kg) resulted in 5 CR and 5 TFS. Treatment with Conjugate 4 (1.0/0.035 mg/kg) resulted in 10 CR and 10 TFS. Treatment with Conjugate 4 (3.0/0.10 mg/kg) resulted in 9 CR and 9 TFS. Treatment with Conjugate 3 (0.30/0.012 mg/kg) and anti-PD-1 RMP1-14 (5 mg) resulted in 1 PR. Treatment with Conjugate 3 (1.0/0.040 mg/kg) and anti-PD-1 RMP1-14 (5 mg) resulted in 8 CR and 8 TFS. Treatment with Conjugate 4 (0.30/0.012 mg/kg) and anti-PD-1 RMP1-14 (5 mg) resulted in 1 PR, 8 CR, and 8 TFS. Treatment with Conjugate 4 (1.0/0.035 mg/kg) and anti-PD-1 RMP1-14 (5 mg) resulted in 10 CR and 10 TFS.

### Example 10: Re-Challenge Study for Tumor Growth Response after Administration of HER2 STING Agonist Antibody-Drug Conjugates in Combination with Anti-PD-1

Tumor-free mice (i.e. mice with complete tumor regressions at end of study) from Example 9 previously inoculated subcutaneously with EMT6-RHER2 MSA on the right flank and treated with a combination of Conjugate 4 (0.30/0.012 mg/kg) and anti-PD-1 RMP1-14 (5 mg/kg), or Conjugate 4 (0.3/0.012 mg/kg) and age-matched untreated animals were inoculated subcutaneously on the left flank with EMT-6-MSA mammary carcinoma cells (5×10⁶ cells per mouse) and on the right flank with CT26 colon/colorectal cancer cells (3×10⁵ cells per mouse). Tumor growth on both flanks was monitored and measured twice weekly and the animals were weighed twice weekly.

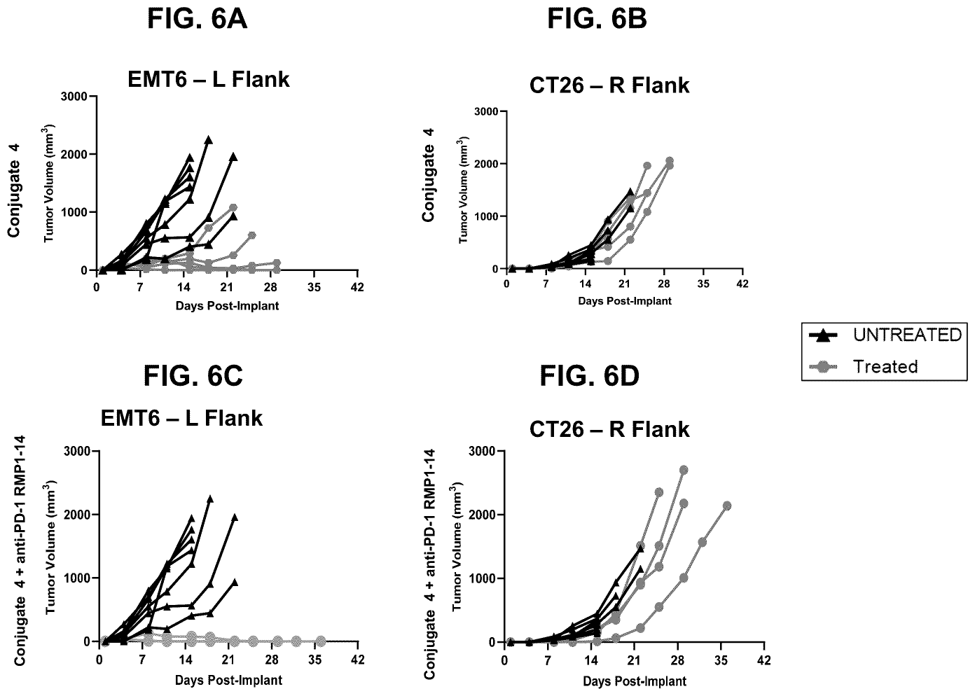

FIGS. 6A and 6B shows the tumor volumes of mice previously treated with Conjugate 4 (0.3/0.012 mg/kg) when rechallenged with EMT-6-MSA cells or CT26 colon/colorectal cancer cells respectively. FIGS. 6C and 6D shows the tumor volumes of mice previously treated with Conjugate 4 (0.30/0.012 mg/kg) and anti-PD-1 RMP1-14 (5 mg/kg) when rechallenged with EMT-6-MSA cells or CT26 colon/colorectal cancer cells respectively. Mice previously treated with Conjugate 4 or Conjugate 4 and anti-PD-1 RMP1-14 (5 mg/kg) when rechallenged with EMT-6-MSA cells resulted in 2 out of 5 tumors rejected and 4 out of 4 tumors rejected respectively. Mice previously treated with Conjugate 4 or Conjugate 4 and anti-PD-1 RMP1-14 when rechallenged with CT26 cells did not reject any cells. Mice treated with Conjugate 4 as a monotherapy or in combination with anti-PD-1 RMP1-14 showed immunological memory.

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A combination therapy comprising at least one HER2-targeted STING agonist antibody-drug conjugate and at least one HER2-targeted therapy or at least one immunotherapy, wherein the HER2-targeted STING agonist antibody-drug conjugate is a conjugate of Formula (A): 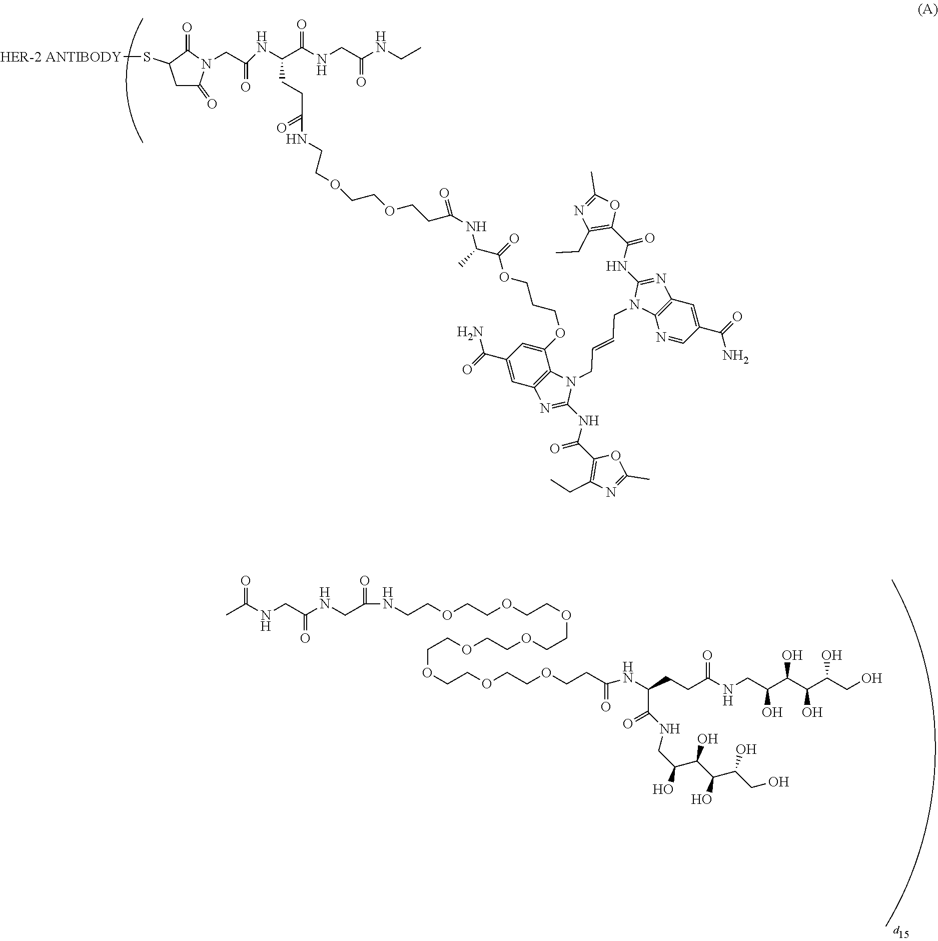
wherein the conjugate comprises a HER2 antibody comprising a variable heavy chain complementarity determining region 1 (CDRH1) comprising the amino acid sequence FTFSSYSMN (SEQ ID NO: 5); a variable heavy chain complementarity determining region 2 (CDRH2) comprising the amino acid sequence YISSSSSTIYYADSVKG (SEQ ID NO: 6); a variable heavy chain complementarity determining region 3 (CDRH3) comprising the amino acid sequence GGHGYFDL (SEQ ID NO: 7); and a variable light chain complementarity determining region 1 (CDRL1) comprising the amino acid sequence RASQSVSSSYLA (SEQ ID NO: 12); a variable light chain complementarity determining region 2 (CDRL2) comprising the amino acid sequence GASSRAT (SEQ ID NO: 13); and a variable light chain complementarity determining region 3 (CDRL3) comprising the amino acid sequence QQYHHSPLT (SEQ ID NO: 14), and d₁₅ is about 8.

2. The conjugate of claim 1, wherein the HER2 antibody specifically binds to an epitope of the human HER2 receptor that includes residues 452 to 531 of the extracellular domain of the human HER2 receptor, residues 474 to 553 of SEQ ID NO: 1 or residues 452 to 531 of SEQ ID NO: 16.

3. The combination of claim 1, comprising at least one HER2-targeted STING agonist antibody-drug conjugate and at least one HER2-targeted therapy.

4. The combination of claim 1, comprising at least one HER2-targeted STING agonist antibody-drug conjugate and at least one immunotherapy.

5. The combination of claim 1, wherein the HER2-targeted STING agonist antibody-drug conjugate enhances the efficacy of the HER2-targeted therapy or the immunotherapy.

6. The combination of claim 3, wherein the HER2-targeted therapy is an antibody or antigen binding fragment thereof that specifically binds HER2, a HER2-targeted antibody-drug conjugate that specifically binds HER2 or a small molecule inhibitor of HER2.

7. The combination of claims 6, wherein the antibody or antigen binding fragment thereof that specifically binds HER2 is a HER2 antibody, a HER2 dimerization inhibitor antibody or a combination thereof.

8. The combination of claim 7, wherein the HER2 antibody or the HER2 dimerization inhibitor antibody is trastuzumab, pertuzumab or margetuximab or biosimilars thereof.

9. The combination of claim 6, wherein HER2-targeted therapy is trastuzumab, pertuzumab, a combination of trastuzumab and pertuzumab, margetuximab, a biosimilar of trastuzumab, a biosimilar of pertuzumab, a combination of a biosimilar of trastuzumab and a biosimilar of pertuzumab, or a biosimilar of margetuximab.

10. The combination of claim 1, comprising at least one HER2-targeted STING agonist antibody-drug conjugate and trastuzumab, pertuzumab, a combination of trastuzumab and pertuzumab, margetuximab, a biosimilar of trastuzumab, a biosimilar of pertuzumab, a combination of a biosimilar of trastuzumab and a biosimilar of pertuzumab or a biosimilar of margetuximab.

11. The combination of claim 6, wherein the HER2-targeted antibody-drug conjugate that specifically binds HER2 is ado-trastuzumab emtansine (T-DM1) or famtrastuzumab deruxtecan (trastuzumab deruxtecan).

12. The combination of claim 1, comprising at least one HER2-targeted STING agonist antibody-drug conjugate and ado-trastuzumab emtansine (T-DM1) or fam-trastuzumab deruxtecan (trastuzumab deruxtecan).

13. The combination of claim 6, where the small molecule inhibitor of HER2 is tucatinib, neratinib or lapatinib.

14. The combination of claim 1, comprising at least one HER2-targeted STING agonist antibody-drug conjugate and tucatinib, neratinib or lapatinib.

15. The combination of claim 4, wherein the immunotherapy is an immune checkpoint inhibitor.

16. The combination of claim 15, wherein the immune checkpoint inhibitor is a monoclonal antibody, a humanized antibody, a fully human antibody, a fusion protein or a combination thereof.

17. The combination of claim 15, wherein the immune checkpoint inhibitor is a PD-1 inhibitor or a PD-L1 inhibitor.

18. The combination of claim 17, wherein the PD-1 inhibitor or the PD-L1 inhibitor is avelumab, durvalumab, dostarlimab, pembrolizumab, cemiplimab, nivolumab, or atezolizumab.

19. The combination of claim 1, comprising at least one HER2-targeted STING agonist antibody-drug conjugate and avelumab, durvalumab, dostarlimab, pembrolizumab, cemiplimab, nivolumab, or atezolizumab.

20. A method for treating, preventing, delaying the progression of or otherwise ameliorating a symptom of a cancer in a subject comprising administering the combination of claim 1.

21. The method of claim 20, wherein the cancer is anal cancer, astrocytoma, leukemia, lymphoma, head and neck cancer, liver cancer, testicular cancer, cervical cancer, sarcoma, hemangioma, esophageal cancer, eye cancer, laryngeal cancer, mouth cancer, mesothelioma, skin cancer, myeloma, oral cancer, rectal cancer, colorectal cancer, throat cancer, bladder cancer, breast cancer, urothelial cancer, uterine cancer, ovarian cancer, prostate cancer, lung cancer, non-small cell lung cancer (NSCLC), colon cancer, pancreatic cancer, renal cancer, gastric cancer or gastric esophagogastric junction cancer.

22. The method of claim 21, wherein the cancer is breast cancer, gastric cancer, gastric esophagogastric junction cancer, colorectal cancer or non-small cell lung cancer.

23. The method of claim 20, wherein the immune checkpoint inhibitor and the conjugate are administered simultaneously.

24. The method of claim 20, wherein the HER2-targeted STING agonist antibody-drug conjugate and the HER2-targeted therapy or the immunotherapy are administered simultaneously.

25. The method of claim 20, wherein the HER2-targeted STING agonist antibody-drug conjugate and the HER2-targeted therapy or the immunotherapy are administered sequentially in either order or in alternation.

26. The method of claim 20, wherein the HER2-targeted STING agonist antibody-drug conjugate is administered prior to the HER2-targeted therapy or the immunotherapy.

27. The method of claim 20, wherein the HER2-targeted STING agonist antibody-drug conjugate is administered after the HER2-targeted therapy or the immunotherapy.

28. The combination of claim 1, wherein the HER2-targeted STING agonist antibody-drug conjugate and the HER2-targeted therapy or the immunotherapy are formulated in the same formulation.

29. The combination of claim 1, wherein the HER2-targeted STING agonist antibody-drug conjugate and the HER2-targeted therapy or the immunotherapy are formulated in separate formulations.

30. A kit comprising the combination of claim 1 and an instruction for administration.


SEQUENCE LISTING (16 sequences)

SEQ ID NO: 1 - protein, 1255 aa, Homo sapiens [sequence omitted]

SEQ ID NO: 2 - protein, 117 aa, Homo sapiens
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARGGHGYFDLWGRGTLVTVSS

SEQ ID NO: 3 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 446 aa, Homo sapiens
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARGGHGYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 9 aa, Homo sapiens
FTFSSYSMN

SEQ ID NO: 6 - protein, 17 aa, Homo sapiens
YISSSSSTIYYADSVKG

SEQ ID NO: 7 - protein, 8 aa, Homo sapiens
GGHGYFDL

SEQ ID NO: 8 - DNA, 351 bp, Homo sapiens
  source 1..351: mol_type = genomic DNA
gaggtgcagctggtggagtctgggggaggcttggtacagcctggggggtccctgagactctcctgtgcagcctctggattcaccttcagtagctatagcatgaactgggtccgccaggctccagggaaggggctggagtgggtttcatacattagtagtagtagtagtaccatatactacgcagactctgtgaagggccgattcaccatctccagagacaatgccaagaactcactgtatctgcaaatgaacagcctgagagctgaggacacggcggtgtactactgcgccagaggtggacacggatatttcgacctatgggggagaggtaccttggtcaccgtctcctca

SEQ ID NO: 9 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYHHSPLTFGGGTKVEIK

SEQ ID NO: 10 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 215 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYHHSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 12 aa, Homo sapiens
RASQSVSSSYLA

SEQ ID NO: 13 - protein, 7 aa, Homo sapiens
GASSRAT

SEQ ID NO: 14 - protein, 9 aa, Homo sapiens
QQYHHSPLT

SEQ ID NO: 15 - DNA, 324 bp, Homo sapiens
  source 1..324: mol_type = genomic DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtaccaccacagtcctctcacttttggcggagggaccaaggttgagatcaaa

SEQ ID NO: 16 - protein, 630 aa, Homo sapiens
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLT